SEQUENCE LISTING (3 sequences)

SEQ ID NO: 1 - protein, 738 aa, Adeno Associated Virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 736 aa, Adeno Associated Virus
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 3 - DNA, 1672 bp, Artificial Sequence
  CAG Promoter Sequence
atagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

(19) United States
(12) Patent Application Publication
(10) Pub. No.: US 2022/0011308 A1
(43) Pub. Date: Jan. 13, 2022

(54) IN VITRO ASSAY FOR DETECTING ENHANCERS AND INHIBITORS OF ADENO ASSOCIATED VIRUS (AAV) VECTOR TRANSDUCTION AND/OR DETECTING OR QUANTITATING ANTI-AAV BINDING ANTIBODIES

(71) Applicant: SPARK THERAPEUTICS, INC., Philadelphia, PA (US)

(72) Inventors: Klaudia KURANDA, Philadelphia, PA (US); Xavier ANGUELA, Barcelona (ES); Federico MINGOZZI, Philadelphia, PA (US)

(73) Assignee: SPARK THERAPEUTICS, INC., Philadelphia, PA (US)

(21) Appl. No.: 17/309,261

(22) PCT Filed: Nov. 15, 2019
(86) PCT No.: PCT/US2019/061851

§ 371 Date: May 12, 2021

Related U.S. Application Data
(60) Provisional application No. 62/768,665, filed on Nov. 16, 2018.

(51) Int. Cl. G01N 33/569; G06F 17/18

(52) U.S. Cl. CPC G01N 33/56983; G01N 2333/075; G06F 17/18

(57) ABSTRACT

Disclosed herein are methods for analyzing for or detecting the presence of non-antibody inhibitors and/or enhancers of adeno-associated virus (AAV) vector cell transduction in a biological sample from a subject. Also disclosed herein are methods for analyzing for, or detecting the presence of, AAV binding antibodies that inhibit, reduce or decrease AAV vector cell transduction in a biological sample from a subject. The methods rely, in part, on the use of empty capsid AAV particles to absorb AAV binding antibodies, to detect enhancers or inhibitors of AAV vector cell transduction, when present, in a biological sample analyzed for AAV neutralizing antibodies (NAbs).

### RELATED APPLICATIONS

This application claims priority to U.S. Provisional Patent Application No. 62/768,665, filed Nov. 16, 2018. The entire contents of the foregoing application are incorporated herein by reference, including all text, tables, sequence listings and drawings.

### INTRODUCTION

Adeno-associated virus (AAV) vector gene transfer has demonstrated clinical efficacy in treatment of Leber congential amaurosis and in human clinical trials for bleeding disorders hemophilia A and B. Due to exposure to wild-type AAV, a variable percent of humans will present with antibodies binding to the capsid, which can inhibit or prevent AAV vector cell transduction. Such antibodies that bind to AAV are a major hurdle to AAV based gene therapy vectors, leaving some patients without access to potentially life-saving therapies. As a result, subjects positive for neutralizing antibodies (NAbs) to AAV are often excluded from enrollment in gene therapy trials and are also less optimal candidates for gene therapy treatment.

Anti-AAV antibodies can be measured with binding assays, in which IgG binding to the virus is detected, or cell transduction inhibition assays, in which the efficiency of cell transduction of a reporter vector is measured in vitro. While antibody binding assays are easy to set up, they do not identify which binding antibodies affect AAV vector transduction. Conversely, cell-based NAb assays do measure the extent of inhibition of vector transduction mediated by anti-AAV circulating factors, but are time consuming and present challenges in terms of both sensitivity and accuracy. In addition, the lack of standardization of procedures for NAb assays is an obstacle for interpretation of results across gene therapy trials.

### SUMMARY

As disclosed herein, there are other factors distinct from AAV binding antibodies that can inhibit, reduce or decrease AAV vector cell transduction. As also disclosed herein, there are factors that can enhance AAV vector cell transduction. These enhancing and inhibiting factors can be present in certain subjects amenable to treatment with AAV based gene therapy or participation in an AAV based gene therapy clinical trial. Typically, subjects are evaluated for the presence of AAV binding antibodies to determine suitability/eligibility for a gene therapy treatment. Subjects can also be evaluated for the presence of AAV binding antibodies after receiving a gene therapy treatment for purposes of monitoring the development of antibodies or a subsequent re-dosing of a gene therapy treatment. However, if a subject in which it is desired to measure AAV binding antibodies has enhancers or inhibitors of AAV vector cell transduction, a typical cell-based antibody assay will provide inaccurate quantitative results in terms of AAV binding antibody titer.

For example, in the case of factors that enhance AAV vector cell transduction, present in a subject having AAV binding antibodies, the amount of AAV binding antibodies would be underestimated, and if low enough can lead to a false negative for AAV binding antibodies in the subject. Such false negative subjects are actually positive for AAV binding antibodies.

In the case of factors that inhibit AAV vector cell transduction, present in a subject, even if there are no detectable AAV binding antibodies, if there are enough inhibitors present to inhibit or reduce AAV vector cell transduction, the test would result in a false positive for AAV binding antibodies in the subject. Such false positive subjects are actually negative for AAV binding antibodies or have a relatively low AAV antibody titer.

Accordingly, the invention provides, inter alia, methods for analyzing a sample for and methods for detecting presence of such enhancer factors or inhibitory factors in a sample, such as a biological sample from a subject.

In certain embodiments, a method for analyzing for or detecting the presence of enhancers of adeno-associated virus (AAV) vector cell transduction in a biological sample from a subject includes:
(a) providing: a. infectious recombinant AAV particles comprising a recombinant AAV vector, the recombinant AAV vector comprising a reporter transgene operably linked to one or more expression regulatory element, b. empty capsid AAV particles, c. cells permissive of AAV infection and d. a biological sample from a subject;
(b) performing a first assay by: a. contacting the cells with the infectious recombinant AAV particles under conditions in which the cells are transduced by the infectious recombinant AAV particles and the reporter transgene is expressed by the cells and b. measuring expression of the reporter transgene and assigning a value denoted MAX;
(c) performing a second assay by: a. mixing the biological sample with the empty capsid AAV particles to produce a mixture (M), and incubating the M under conditions allowing the empty capsid AAV particles to bind to any AAV binding antibodies present in the biological sample, b. contacting the cells with the M and the infectious recombinant AAV particles, under conditions in which the cells are transduced by the infectious recombinant AAV particles and the reporter transgene is expressed by the cells and c. measuring expression of the reporter transgene and assigning a value denoted S.EV; and
(d) comparing the S.EV to the MAX, wherein if the S.EV is greater than the MAX the biological sample from the subject contains enhancers of AAV vector cell transduction.

In certain embodiments, a method for analyzing for or detecting the presence of inhibitors of adeno-associated virus (AAV) vector cell transduction in a biological sample from a subject includes:
(a) providing: a. infectious recombinant AAV particles comprising a recombinant AAV vector, the recombinant AAV vector comprising a reporter transgene operably linked to one or more expression regulatory element, b. empty capsid AAV particles, c. cells permissive of AAV infection and d. a biological sample from a subject;
(b) performing a first assay by: a. contacting the cells with the infectious recombinant AAV particles under conditions in which the cells are transduced by the infectious recombinant AAV particles and the reporter transgene is expressed by the cells and b. measuring expression of the reporter transgene and assigning a value denoted MAX;
(c) performing a second assay by: a. mixing the biological sample with the empty capsid AAV particles to produce a mixture (M), and incubating the M under conditions allowing the empty capsid AAV particles to bind to any AAV binding antibodies present in the biological sample, b. contacting the cells with the M and the infectious recombinant AAV particles, under conditions in which the cells are transduced by the infectious recombinant AAV particles and the reporter transgene is expressed by the cells and c. measuring expression of the reporter transgene and assigning a value denoted S.EV; and
(d) comparing the S.EV to the MAX, wherein if the S.EV is less than the MAX the biological sample from the subject contains inhibitors of AAV vector cell transduction.

In certain embodiments, a method for analyzing for or detecting the presence of enhancers of adeno-associated virus (AAV) vector cell transduction in a biological sample from a subject includes:
(a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element and (iv) the reporter transgene is flanked by one or more flanking element;
(b) providing cells that can be infected with the infectious recombinant AAV particles;
(c) contacting the cells of (b) with the infectious recombinant AAV particles of (a) under conditions in which the cells of (b) are transduced by the infectious recombinant AAV particles of (a) and the reporter transgene is expressed by the cells of (b);
(d) measuring expression of the reporter transgene and assigning a value denoted MAX that reflects the amount of reporter transgene expression of (c);
(e) providing infectious recombinant AAV particles (a);
(f) providing a biological sample from a subject;
(g) providing cells that can be infected with the infectious recombinant AAV particles;
(h) mixing the biological sample of (f) with empty capsid AAV particles to produce a mixture (M) and incubating the M under conditions allowing the empty capsid AAV particles to bind to any AAV binding antibodies present in the biological sample;
(i) contacting the cells of (g) with the M and the infectious recombinant AAV particles of (e) under conditions in which the infectious recombinant AAV particles of (e) can transduce the cells of (g) and express the reporter transgene in the cells of (g);
(j) measuring expression of the reporter transgene and assigning a value denoted S.EV that reflects the amount of reporter transgene expression of (i); and
(k) comparing the S.EV to the MAX, wherein if the S.EV is greater than the MAX the biological sample from the subject contains enhancers of AAV vector cell transduction.

In certain embodiments, a method for analyzing for or detecting the presence of inhibitors of adeno-associated virus (AAV) vector cell transduction in a biological sample from a subject includes: (a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element and (iv) the reporter transgene is flanked by one or more flanking element;
(b) providing cells that can be infected with the infectious recombinant AAV particles;
(c) contacting the cells of (b) with the infectious recombinant AAV particles of (a) under conditions in which the cells of (b) are transduced by the infectious recombinant AAV particles of (a) and the reporter transgene is expressed by the cells of (b);
(d) measuring expression of the reporter transgene and assigning a value denoted MAX that reflects the amount of reporter transgene expression of (c);
(e) providing infectious recombinant AAV particles (a);
(f) providing a biological sample from a subject;
(g) providing cells that can be infected with the infectious recombinant AAV particles;
(h) mixing the biological sample of (f) with empty capsid AAV particles to produce a mixture (M) and incubating the M under conditions allowing the empty capsid AAV particles to bind to any AAV binding antibodies present in the biological sample;
(i) contacting the cells of (g) with the M and the infectious recombinant AAV particles of (e) under conditions allowing the infectious recombinant AAV particles of (e) to transduce the cells of (g) and express the reporter transgene in the cells of (g);
(j) measuring expression of the reporter transgene and assigning a value denoted S.EV that reflects the amount of reporter transgene expression of (i);
(k) comparing the S.EV to the MAX, wherein if the S.EV is less than the MAX the biological sample from the subject contains inhibitors of AAV vector cell transduction, expression or secretion of a protein encoded by the vector.

In certain embodiments, a method for analyzing for or detecting the presence of enhancers of adeno-associated virus (AAV) vector cell transduction in a biological sample from a subject includes:
(a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element and (iv) the reporter transgene is flanked by one or more flanking element;
(b) providing cells that can be infected with the infectious recombinant AAV particles;
(c) providing a biological sample from a subject;
(d) mixing the biological sample of (c) with empty capsid AAV particles to produce a mixture (M) and incubating the M under conditions allowing the empty capsid AAV particles to bind to any AAV binding antibodies present in the biological sample;
(e) contacting the cells of (b) with the M and the infectious recombinant AAV particles of (a) under conditions allowing the infectious recombinant AAV particles of (a) to transduce the cells of (b) and express the reporter transgene in the cells of (b);
(f) measuring expression of the reporter transgene;
(g) comparing the expression of (f) to a positive (+) control, wherein the + control is the expression of the reporter transgene without the sample from the subject and without addition of the empty capsid AAV particles, wherein if expression of (f) is greater than the + control the biological sample from the subject contains enhancers of AAV vector cell transduction.

In certain embodiments, a method for analyzing for or detecting the presence of inhibitors of adeno-associated virus (AAV) vector cell transduction in a biological sample from a subject includes:
(a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element, and (iv) the reporter transgene is flanked by one or more flanking element;
(b) providing cells that can be infected with the infectious recombinant AAV particles;
(c) providing a biological sample from a subject;
(d) mixing the biological sample of (c) with empty capsid AAV particles to produce a mixture (M) and incubating the M under conditions allowing the empty capsid AAV particles to bind to any AAV binding antibodies present in the biological sample;
(e) contacting the cells of (b) with the M and the infectious recombinant AAV particles of (a) under conditions allowing the infectious recombinant AAV particles of (a) to transduce the cells of (b) and express the reporter transgene in the cells of (b);
(f) measuring expression of the reporter transgene;
(g) comparing the expression of (f) to a positive (+) control, wherein the + control is the expression of the reporter transgene without the sample from the subject and without addition of the empty capsid AAV particles, wherein if expression of (f) is less than the + control the biological sample from the subject contains inhibitors of AAV vector cell transduction.

The invention also provides, inter alia, methods for analyzing for, detecting or quantifying AAV binding antibodies that inhibit, reduce or decrease AAV vector cell transduction in a sample, such as a biological sample from a subject.

In certain embodiments, a method for analyzing for, detecting or quantifying AAV binding antibodies that inhibit AAV vector cell transduction in a biological sample from a subject includes:
(a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element and (iv) the reporter transgene is flanked by one or more flanking element;
(b) providing cells that can be infected with the infectious recombinant AAV particles;
(c) contacting the cells of (b) with the infectious recombinant AAV particles of (a) under conditions in which the cells of (b) are transduced by the infectious recombinant AAV particles of (a) and the reporter transgene is expressed by the cells of (b);
(d) measuring expression of the reporter transgene and assigning a value denoted MAX that reflects the amount of reporter transgene expression of (c);
(e) providing infectious recombinant AAV particles (a);
(f) providing a diluted biological sample from a subject;
(g) providing cells that can be infected with the infectious recombinant AAV particles;
(h) mixing the diluted biological sample of (f) with empty capsid AAV particles to produce a mixture (M) and incubating the M under conditions allowing the empty capsid AAV particles to bind to any AAV binding antibodies present in the biological sample;
(i) contacting the cells of (g) with the M and the infectious recombinant AAV particles of (e) under conditions allowing the infectious recombinant AAV particles of (e) to transduce the cells of (g) and express the reporter transgene in the cells of (g);
(j) measuring expression of the reporter transgene and assigning a value denoted S.EV that reflects the amount of reporter transgene expression of (i);
(k) providing infectious recombinant AAV particles (a);
(l) providing a diluted biological sample from the same subject that provided the sample of (f)
(m) providing cells that can be infected with the infectious recombinant AAV particles;
(n) mixing the diluted biological sample of (l) with the infectious recombinant AAV particles of (k) to produce a mixture (M);
(o) contacting the cells of (m) with the M under conditions allowing the infectious recombinant AAV particles of (k) to transduce the cells of (m) and express the reporter transgene in the cells of (m);
(p) measuring expression of the reporter transgene and assigning a value denoted S that reflects the amount of reporter transgene expression of (o);
(q) performing steps (h)-(j) and (n)-(p) at least twice at different sample dilutions;
(r) wherein if S is less than MAX and S.EV is equal to or greater than MAX, AAV binding antibodies that inhibit AAV vector cell transduction are present in the diluted sample; and, optionally
(s) measuring expression of a negative control of cells that can be infected with the infectious recombinant AAV particles (a) but are not infected with infectious recombinant AAV particles (a) to provide a background value denoted MIN, wherein MIN may be subtracted from any one of S, MAX and/or S.EV.

In certain embodiments method steps can be performed in any suitable order unless otherwise indicated herein.

In certain embodiments, a method further includes after step (r) or after step (s), calculating the titer of AAV binding antibodies in the biological sample, the titer corresponding to the lowest dilution of the biological sample that provides about 50% or more inhibition of reporter transgene expression, wherein if the lowest dilution that provides about 50% or more inhibition of reporter transgene expression is greater than or equal to about 1:5, the titer is the dilution that provides the about 50% or more inhibition determined by the formula S/MAX, or wherein if the lowest dilution that provides about 50% or more inhibition of reporter transgene expression is less than about 1:5, the titer is the dilution that provides the about 50% or more inhibition determined by the formula S/S.EV.

In certain embodiments, a method further includes step (t), calculating the titer of AAV binding antibodies in the biological sample, the titer corresponding to the lowest dilution of the biological sample that provides about 50% or more inhibition of reporter transgene expression, wherein if the lowest dilution that provides about 50% or more inhibition of reporter transgene expression is greater than or equal to about 1:5, the titer is the dilution that provides the about 50% or more inhibition determined by the formula 100−[[S−MIN)/(MAX−MIN)]×100], or wherein if the lowest dilution that provides about 50% or more inhibition of reporter transgene expression is less than about 1:5, the titer is the dilution that provides the about 50% or more inhibition determined by the formula 100−[[(S−MIN)/(S.EV−MIN)]×100].

In certain embodiments, a method further includes: providing infectious recombinant AAV particles (a); providing cells that can be infected with the infectious recombinant AAV particles; providing empty capsid AAV particles; contacting the cells with the provided empty capsid AAV particles; contacting the cells that have been contacted with the empty capsid AAV particles with the infectious recombinant AAV particles of (a) under conditions in which the cells of (b) are transduced by the infectious recombinant AAV particles of (a) and the reporter transgene is expressed by the cells; and measuring expression of the reporter transgene and assigning a value denoted MAX.EV that reflects the amount of reporter transgene expression.

In certain embodiments, a method further includes calculating a signal-to-noise ratio, denoted S/N, wherein the S/N equals MAX/MIN.

In certain embodiments, a method further includes calculating a percent coefficient of variation (% CV), wherein % CV=(standard deviation/mean)×100%.

In certain embodiments, a method further includes calculating EV interference, wherein EV interference=MAX/MAX.EV.

In certain embodiments, a method further includes (a) measuring expression of the reporter transgene under control conditions comprising one or more dilutions of AAV binding antibodies that bind to AAV vector, and assigning a value denoted HQC to the expression measurement of the dilution that provides a preselected amount of reporter transgene expression relative to MAX or MAX−MIN; and/or (b) measuring expression of the reporter transgene under control conditions comprising the dilution of step (a) that provides the preselected amount of reporter transgene expression relative to MAX or MAX−MIN in the presence of empty capsid AAV particles and assigning a value denoted HQC.EV.

A preselected amount of reporter transgene expression of step (a) can be equal to or less than about 75% of MAX or MAX−MIN, for example and without limitation, equal to or less than about 60% of MAX or MAX−MIN, equal to or less than about 50% of MAX or MAX−MIN, equal to or less than about 40% of MAX or MAX−MIN, equal to or less than about 30% of MAX or MAX−MIN. In certain embodiments, a preselected amount of reporter transgene expression of step (a) is equal to or less than about 25% of MAX or MAX−MIN. In certain embodiments, a preselected amount of reporter transgene expression of step (a) is equal to or less than about 20% of MAX or MAX−MIN.

In certain embodiments, a method further includes calculating EV efficacy, wherein EV efficacy=HQC.EV/HQC.

In certain embodiments, steps (h)-(j) and (n)-(p) are performed at least 3, 4, 5 or 6 times at 3, 4, 5 or 6 different dilutions of the biological sample.

In certain embodiments, a sample is diluted between about 1:1 and about 1:5000 prior to contacting or incubating with the infectious recombinant AAV particles of (a), (e) or (k).

In certain embodiments, a sample is diluted between about 1:1 and about 1:1000 prior to contacting or incubating with the infectious recombinant AAV particles of (a), (e) or (k).

In certain embodiments, a sample is diluted between about 1:1 and about 1:500 prior to contacting or incubating with the infectious recombinant AAV particles of (a), (e) or (k).

In certain embodiments, a sample is diluted between about 1:1 and about 1:100 prior to contacting or incubating with the infectious recombinant AAV particles of (a), (e) or (k).

In certain embodiments, steps (h)-(j) and (n)-(p) are performed at about 1:1, about 1:2.5, about 1:5, about 1:10, about 1:100 and/or about 1:1000 dilutions of the biological sample.

In certain embodiments, a method is completed within about 48 hours of when step (c) or (d) begins or when step (c) or (d) is completed.

In certain embodiments, cells that can be infected with the infectious recombinant AAV particles of any of steps (c), (e), (i), or (o) are contacted within about 2 hours after the cells that can be infected with the infectious recombinant AAV particles are thawed from freezing.

In certain embodiments, cells that can be infected with the infectious recombinant AAV particles of any of steps (c), (e), (i), or (o) are contacted within about 1 hour after the cells that can be infected with the infectious recombinant AAV particles are thawed from freezing.

In certain embodiments, cells that can be infected with the infectious recombinant AAV particles of any of steps (c), (e), (i), or (o) are at least about 60% confluent.

In certain embodiments, cells that can be infected with the infectious recombinant AAV particles of any of steps (c), (e), (i), or (o) are at least about 70% confluent.

In certain embodiments, cells that can be infected with the infectious recombinant AAV particles of any of steps (c), (e), (i), or (o) are at least about 80% confluent.

In certain embodiments, cells that can be infected with the infectious recombinant AAV particles of any of steps (c), (e), (i), or (o) are at least about 90% confluent.

The invention also provides, inter alia, carriers and plates having one or more components used in the invention methods. Carriers and plates can include, for example and without limitation, cells that can be infected with infectious recombinant AAV particles, a biological sample from a subject such as a diluted biological sample from a subject and/or empty capsid AAV particles.

In certain embodiments, a carrier or plate has individually disposed thereon:
(a) cells that can be infected with infectious recombinant AAV particles comprising a reporter transgene the infectious recombinant AAV particles comprising the reporter transgene;
(b) a diluted biological sample from a subject, cells that can be infected with an infectious recombinant AAV particles and empty capsid AAV particles; and/or
(c) a diluted biological sample from the same subject that provided the sample of (b), cells that can be infected with infectious recombinant AAV particles comprising a reporter transgene and the infectious recombinant AAV particles comprising the reporter transgene.

In certain embodiments, each of the components on a carrier or plate can be within individual wells. In certain embodiments, each of the components on a carrier or plate can be in the amounts disclosed for the methods herein.

In certain embodiments, a carrier or plate has each of (a), (b) and (c) components disposed within a separate tube or a separate single well of a multiwell carrier or plate.

In certain embodiments, a carrier or plate comprises plastic or glass.

In certain embodiments, a carrier or plate is a multiwell plate or dish.

As described herein, in certain embodiments, a method for analyzing for or detecting the presence of enhancers of adeno-associated virus (AAV) vector cell transduction in a biological sample from a subject includes:
(a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element; and (iv) the reporter transgene is flanked by one or more flanking element;
(b) providing cells that can be infected with said infectious recombinant AAV particles;
(c) contacting the cells of (b) with the infectious recombinant AAV particles of (a) under conditions in which the cells of (b) are transduced by the infectious recombinant AAV particles of (a) and the reporter transgene is expressed by said cells of (b);
(d) measuring expression of the reporter transgene and assigning a value denoted MAX that reflects the amount of reporter transgene expression of (c);
(e) providing infectious recombinant AAV particles (a);
(f) providing a biological sample from a subject;
(g) providing cells that can be infected with said infectious recombinant AAV particles;
(h) mixing the biological sample of (f) with empty capsid AAV particles to produce a mixture (M) and incubating said M under conditions allowing the empty capsid AAV particles to bind to any AAV binding antibodies present in the biological sample;
(i) contacting said cells of (g) with said M and said infectious recombinant AAV particles of (e) under conditions in which the infectious recombinant AAV particles of (e) can transduce said cells of (g) and express the reporter transgene in said cells of (g);
(j) measuring expression of the reporter transgene and assigning a value denoted S.EV that reflects the amount of reporter transgene expression of (i); and
(k) comparing said S.EV to said MAX, wherein if said S.EV is greater than said MAX the biological sample from the subject contains enhancers of AAV vector cell transduction.

In certain embodiments, one or more of steps (c), (d), (h), (i), (j), or (k) may be performed with an automated system.

In certain embodiments, the automated system comprises contacting components, measuring components, mixing components, incubating components, a processor, and non-transitory electronic storage. The non-transitory electronic storage is configured to cause the processor to control the contacting components, the measuring components, the mixing components, and the incubating components. The method further comprises:
(c) contacting, with the contacting components, the cells of (b) with the infectious recombinant AAV particles of (a) under the conditions in which the cells of (b) are transduced by the infectious recombinant AAV particles of (a) and the reporter transgene is expressed by said cells of (b);
(d) measuring, with the measuring components, the expression of the reporter transgene, assigning, with the processor, the value denoted MAX that reflects the amount of reporter transgene expression of (c), and storing, with the processor, the value denoted MAX in the non-transitory electronic storage;
(h) mixing, with the mixing components, the biological sample of (f) with the empty capsid AAV particles to produce the mixture M and incubating, with the incubating components, said M under the conditions allowing the empty capsid AAV particles to bind to any AAV binding antibodies present in the biological sample that inhibits, reduces or decreases AAV vector cell transduction;
(i) contacting, with the contacting components, said cells of (g) with said M and said infectious recombinant AAV particles of (e) under the conditions in which the infectious recombinant AAV particles of (e) can transduce said cells of (g) and express the reporter transgene in said cells of (g);
(j) measuring, with the measuring components, the expression of the reporter transgene, assigning, with the processor, the value denoted S.EV that reflects the amount of reporter transgene expression of (i), and storing, with the processor, the value denoted S.EV in the non-transitory electronic storage; and
(k) comparing, with the processor, said S.EV to said MAX, wherein if said S.EV is greater than said MAX the processor determines the biological sample from the subject contains enhancers of AAV vector cell transduction, and optionally outputting, with the processor, an indication that the biological sample from the subject contains the enhancers for display.

As described herein, in certain embodiments, a method for analyzing for or detecting the presence of inhibitors of adeno-associated virus (AAV) vector cell transduction in a biological sample from a subject includes:
(a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element; and (iv) the reporter transgene is flanked by one or more flanking element;
(b) providing cells that can be infected with said infectious recombinant AAV particles;
(c) contacting the cells of (b) with the infectious recombinant AAV particles of (a) under conditions in which the cells of (b) are transduced by the infectious recombinant AAV particles of (a) and the reporter transgene is expressed by said cells of (b);
(d) measuring expression of the reporter transgene and assigning a value denoted MAX that reflects the amount of reporter transgene expression of (c);
(e) providing infectious recombinant AAV particles (a);
(f) providing a biological sample from a subject;
(g) providing cells that can be infected with said infectious recombinant AAV particles;
(h) mixing the biological sample of (f) with empty capsid AAV particles to produce a mixture (M) and incubating said M under conditions allowing the empty capsid AAV particles to bind to any AAV binding antibodies present in the biological sample;
(i) contacting said cells of (g) with said M and said infectious recombinant AAV particles of (e) under conditions allowing the infectious recombinant AAV particles of (e) to transduce said cells of (g) and express the reporter transgene in said cells of (g);
(j) measuring expression of the reporter transgene and assigning a value denoted S.EV that reflects the amount of reporter transgene expression of (i); and
(k) comparing said S.EV to said MAX, wherein if said S.EV is less than said MAX the biological sample from the subject contains inhibitors of AAV vector cell transduction, expression or secretion of a protein encoded by the vector.

In certain embodiments, one or more of steps (c), (d), (h), (i), (j), or (k) may be performed with an automated system.

In certain embodiments, the automated system comprises contacting components, measuring components, mixing components, incubating components, a processor, and non-transitory electronic storage. The non-transitory electronic storage is configured to cause the processor to control the contacting components, the measuring components, the mixing components, and the incubating components. The method further comprises:
(c) contacting, with the contacting components, the cells of (b) with the infectious recombinant AAV particles of (a) under conditions in which the cells of (b) are transduced by the infectious recombinant AAV particles of (a) and the reporter transgene is expressed by said cells of (b);
(d) measuring, with the measuring components, expression of the reporter transgene, assigning, with the processor, the value denoted MAX that reflects the amount of reporter transgene expression of (c), and storing, with the processor, the value denoted MAX in the non-transitory electronic storage;
(h) mixing, with the mixing components, the biological sample of (f) with the empty capsid AAV particles to produce the mixture M and incubating, with the incubating components, said M under conditions allowing the empty capsid AAV particles to bind to any AAV binding antibodies present in the biological sample that inhibits, reduces or decreases AAV vector cell transduction;
(i) contacting, with the contacting components, said cells of (g) with said M and said infectious recombinant AAV particles of (e) under conditions allowing the infectious recombinant AAV particles of (e) to transduce said cells of (g) and express the reporter transgene in said cells of (g);
(j) measuring, with the measuring components, expression of the reporter transgene, assigning, with the processor, the value denoted S.EV that reflects the amount of reporter transgene expression of (i), and storing, with the processor, the value denoted S.EV in the non-transitory electronic storage; and
(k) comparing, with the processor, said S.EV to said MAX, wherein if said S.EV is less than said MAX the processor determines the biological sample from the subject contains inhibitors of AAV vector cell transduction, expression or secretion of a protein encoded by the vector, and optionally outputting, with the processor, an indication that the biological sample from the subject contains the inhibitors for display.

As described herein, in certain embodiments, a method for analyzing for or detecting the presence of enhancers of adeno-associated virus (AAV) vector cell transduction in a biological sample from a subject includes:
(a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element; and (iv) the reporter transgene is flanked by one or more flanking element;
(b) providing cells that can be infected with said infectious recombinant AAV particles;
(c) providing a biological sample from a subject;
(d) mixing the biological sample of (c) with empty capsid AAV particles to produce a mixture (M) and incubating said M under conditions allowing the empty capsid AAV particles to bind to any AAV binding antibodies present in the biological sample;
(e) contacting said cells of (b) with said M and said infectious recombinant AAV particles of (a) under conditions allowing the infectious recombinant AAV particles of (a) to transduce said cells of (b) and express the reporter transgene in said cells of (b);
(f) measuring expression of the reporter transgene; and
(g) comparing said expression of (f) to a positive (+) control, wherein the + control is the expression of the reporter transgene without the sample from the subject and without addition of the empty capsid AAV particles, wherein if expression of (f) is greater than said + control the biological sample from the subject contains enhancers of AAV vector cell transduction.

In certain embodiments, one or more of steps (d), (e), (f), or (g) may be performed with an automated system.

In certain embodiments, the automated system comprises contacting components, measuring components, mixing components, incubating components, a processor, and non-transitory electronic storage. The non-transitory electronic storage is configured to cause the processor to control the contacting components, the measuring components, the mixing components, and the incubating components. The method further comprises:
(d) mixing, with the mixing components, the biological sample of (c) with the empty capsid AAV particles to produce the mixture M and incubating, with the incubating components, said M under the conditions allowing the empty capsid AAV particles to bind to any AAV binding antibodies present in the biological sample that inhibits, reduces or decreases AAV vector cell transduction;
(e) contacting, with the contacting components, said cells of (b) with said M and said infectious recombinant AAV particles of (a) under the conditions allowing the infectious recombinant AAV particles of (a) to transduce said cells of (b) and express the reporter transgene in said cells of (b);
(f) measuring, with the measuring components, expression of the reporter transgene; and
(g) comparing, with the processor, said expression of (f) to the positive (+) control, wherein if said expression of (f) is greater than said + control the processor determines the biological sample from the subject contains enhancers of AAV vector cell transduction, expression or secretion of a protein encoded by the vector, and optionally outputting, with the processor, an indication that the biological sample from the subject contains the enhancers for display.

As described herein, in certain embodiments, a method for analyzing for or detecting the presence of inhibitors of adeno-associated virus (AAV) vector cell transduction in a biological sample from a subject includes:
(a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element, and (iv) the reporter transgene is flanked by one or more flanking element;
(b) providing cells that can be infected with said infectious recombinant AAV particles;
(c) providing a biological sample from a subject;
(d) mixing the biological sample of (c) with empty capsid AAV particles to produce a mixture (M) and incubating said M under conditions allowing the empty capsid AAV particles to bind to any AAV binding antibodies present in the biological sample;
(e) contacting said cells of (b) with said M and said infectious recombinant AAV particles of (a) under conditions allowing the infectious recombinant AAV particles of (a) to transduce said cells of (b) and express the reporter transgene in said cells of (b);
(f) measuring expression of the reporter transgene; and
(g) comparing said expression of (f) to a positive (+) control, wherein the + control is the expression of the reporter transgene without the sample from the subject and without addition of the empty capsid AAV particles, wherein if expression of (f) is less than said + control the biological sample from the subject contains inhibitors of AAV vector cell transduction.

In certain embodiments, one or more of steps (d), (e), (f), or (g) may be performed with an automated system.

In certain embodiments, the automated system comprises contacting components, measuring components, mixing components, incubating components, a processor, and non-transitory electronic storage. The non-transitory electronic storage is configured to cause the processor to control the contacting components, the measuring components, the mixing components, and the incubating components. The method further comprises:
(d) mixing, with the mixing components, the biological sample of (c) with the empty capsid AAV particles to produce the mixture M and incubating, with the incubating components, said M under conditions allowing the empty capsid AAV particles to bind to any AAV binding antibodies present in the biological sample that inhibits, reduces or decreases the AAV vector cell transduction;
(e) contacting, with the contacting components, said cells of (b) with said M and said infectious recombinant AAV particles of (a) under the conditions allowing the infectious recombinant AAV particles of (a) to transduce said cells of (b) and express the reporter transgene in said cells of (b);
(f) measuring, with the measuring components, expression of the reporter transgene; and
(g) comparing, with the processor, said expression of (f) to the positive (+) control, wherein if said expression of (f) is less than said + control the processor determines the biological sample from the subject contains the inhibitors of AAV vector cell transduction, expression or secretion of a protein encoded by the vector, and optionally outputting, with the processor, an indication that the biological sample from the subject contains the inhibitors for display.

As described herein, in certain embodiments, a method for analyzing for, detecting or quantifying AAV binding antibodies that inhibit AAV vector cell transduction in a biological sample from a subject includes:
(a) providing infectious recombinant AAV particles comprising a recombinant AAV vector, wherein (i) the vector comprises a reporter transgene, (ii) the reporter transgene comprises a single-stranded or a self-complementary genome, (iii) the reporter transgene is operably linked to one or more expression regulatory element; and (iv) the reporter transgene is flanked by one or more flanking element;
(b) providing cells that can be infected with said infectious recombinant AAV particles;
(c) contacting the cells of (b) with the infectious recombinant AAV particles of (a) under conditions in which the cells of (b) are transduced by the infectious recombinant AAV particles of (a) and the reporter transgene is expressed by said cells of (b);
(d) measuring expression of the reporter transgene and assigning a value denoted MAX that reflects the amount of reporter transgene expression of (c);
(e) providing infectious recombinant AAV particles (a);
(f) providing a diluted biological sample from a subject;
(g) providing cells that can be infected with said infectious recombinant AAV particles;
(h) mixing the diluted biological sample of (f) with empty capsid AAV particles to produce a mixture (M) and incubating said M under conditions allowing the empty capsid AAV particles to bind to any AAV binding antibodies present in the biological sample;
(i) contacting said cells of (g) with said M and said infectious recombinant AAV particles of (e) under conditions allowing said infectious recombinant AAV particles of (e) to transduce said cells of (g) and express the reporter transgene in said cells of (g);
(j) measuring expression of said reporter transgene and assigning a value denoted S.EV that reflects the amount of reporter transgene expression of (i);
(k) providing infectious recombinant AAV particles (a);
(l) providing a diluted biological sample from the same subject that provided the sample of (f);
(m) providing cells that can be infected with said infectious recombinant AAV particles;
(n) mixing said diluted biological sample of (l) with said infectious recombinant AAV particles of (k) to produce a mixture (M);
(o) contacting said cells of (m) with said M under conditions allowing said infectious recombinant AAV particles of (k) to transduce said cells of (m) and express said reporter transgene in said cells of (m);
(p) measuring expression of said reporter transgene and assigning a value denoted S that reflects the amount of reporter transgene expression of (o);
(q) performing steps (h)-(j) and (n)-(p) at least twice at different sample dilutions;
(r) wherein if S is less than MAX and S.EV is equal to or greater than MAX, AAV binding antibodies that inhibit AAV vector cell transduction are present in said diluted sample; and, optionally
(s) measuring expression of a negative control of cells that can be infected with said infectious recombinant AAV particles (a) but are not infected with infectious recombinant AAV particles (a) to provide a background value denoted MIN, wherein MIN may be subtracted from any one of S, MAX and/or S.EV.

In certain embodiments, one or more of steps (c), (d), (h), (i), (j), (n), (o), (p), or (s) may be performed with an automated system.

In certain embodiments, the automated system comprises contacting components, measuring components, mixing components, incubating components, a processor, and non-transitory electronic storage. The non-transitory electronic storage is configured to cause the processor to control the contacting components, the measuring components, the mixing components, and the incubating components. The method further comprises:
(c) contacting, with the contacting components, the cells of (b) with the infectious recombinant AAV particles of (a) under the conditions in which the cells of (b) are transduced by the infectious recombinant AAV particles of (a) and the reporter transgene is expressed by said cells of (b);
(d) measuring, with the measuring components, the expression of the reporter transgene, assigning, with the processor, the value denoted MAX that reflects the amount of reporter transgene expression of (c), and storing, with the processor, the value denoted MAX in the non-transitory electronic storage;
(h) mixing, with the mixing components, the diluted biological sample of (f) with the empty capsid AAV particles to produce the mixture M and incubating, with the incubating components, said M under the conditions allowing the empty capsid AAV particles to bind to any AAV binding antibodies present in the biological sample that inhibits, reduces or decreases AAV vector cell transduction;
(i) contacting, with the contacting components, said cells of (g) with said M and said infectious recombinant AAV particles of (e) under the conditions allowing the infectious recombinant AAV particles of (e) to transduce said cells of (g) and express the reporter transgene in said cells of (g);
(j) measuring, with the measuring components, the expression of the reporter transgene, assigning, with the processor, the value denoted S.EV that reflects the amount of reporter transgene expression of (i), and storing, with the processor, the value denoted S.EV in the non-transitory electronic storage;
(n) mixing, with the mixing components, the diluted biological sample of (l) with the infectious recombinant AAV particles of (e) to produce the mixture M;

(o) contacting, with the contacting components, said cells of (m) with said M under the conditions allowing the infectious recombinant AAV particles of (k) to transduce said cells of (m) and express the reporter transgene in said cells of (m);

(p) measuring, with the measuring components, expression of the reporter transgene, assigning, with the processor, the value denoted S that reflects the amount of reporter transgene expression of (o), and storing, with the processor, the value denoted S in the non-transitory electronic storage; and

(s) optionally measuring, with the measuring components, expression of the negative control of cells that can be infected with said infectious recombinant AAV particles (a) but are not infected with infectious recombinant AAV particles (a) to provide a background value denoted MIN, wherein MIN may be subtracted, by the processor, from any one of S, MAX and/or S.EV.

In certain embodiments, the method may further comprise step (s) or (t), calculating, with the processor, a titer of the AAV binding antibodies, said titer corresponding to the lowest dilution of the biological sample that provides about 50% or more inhibition of reporter transgene expression, wherein the processor is configured such that if the lowest dilution that provides about 50% or more inhibition of reporter transgene expression is greater or equal to about 1:5, the titer is determined by the formula S/MAX, or if the lowest dilution that provides about 50% or more inhibition of reporter transgene expression titer is less than about 1:5 then the titer is determined by the formula S/S.EV; and optionally outputting, with the processor, an indication of the titer for display.

In certain embodiments, the method may further comprise step (t), calculating, with the processor, a titer of the AAV binding antibodies, said titer corresponding to the lowest dilution of the biological sample that provides about 50% or more inhibition of reporter transgene expression, wherein the processor is configured such that if the lowest dilution that provides about 50% or more inhibition of reporter transgene expression is greater or equal to about 1:5, the titer is determined by the formula 100−[[(S−MIN)/(MAX−MIN)]×100], or if the lowest dilution that provides about 50% or more inhibition of reporter transgene expression titer is less than about 1:5 than the titer is determined by the formula 100−[[(S−MIN)/(S.EV−MIN)]×100]; and optionally outputting, with the processor, an indication of the titer for display.

In certain embodiments, the method further comprises providing infectious recombinant AAV particles (a); providing cells that can be infected with said infectious recombinant AAV particles; providing empty capsid AAV particles; contacting, with the contacting components, said cells with the provided empty capsid AAV particles; contacting, with the contacting components, said cells that have been contacted with the empty capsid AAV particles with the infectious recombinant AAV particles of (a) under conditions in which the cells of (b) are transduced by the infectious recombinant AAV particles of (a) and the reporter transgene is expressed by said cells; and measuring, with the measuring components, expression of the reporter transgene, assigning, with the processor, a value denoted MAX.EV that reflects the amount of reporter transgene expression, and storing, with the processor, the value denoted MAX.EV in the non-transitory electronic storage. In certain embodiments, the method further comprises calculating, with the processor, a signal-to-noise ratio, denoted S/N, wherein the S/N equals MAX/MIN, storing, with the processor, S/N in the non-transitory electronic storage, and optionally outputting, with the processor, an indication of S/N for display.

In certain embodiments, the method further comprises calculating, with the processor, a percent coefficient of variation (% CV), wherein % CV=(standard deviation/mean)×100%, storing, with the processor, % CV in the non-transitory electronic storage, and optionally outputting, with the processor, an indication of % CV for display.

In certain embodiments, the method further comprises calculating, with the processor, EV interference, wherein EV interference=MAX/MAX.EV, storing, with the processor, EV interference in the non-transitory electronic storage, and optionally outputting, with the processor, an indication of EV interference for display.

In certain embodiments, the method further comprises calculating, with the processor, HQC based upon the expression measurement of the dilution that provides a preselected amount of reporter transgene expression relative to MAX or MAX−MIN and/or HQC EV based upon expression of said reporter transgene under control conditions comprising the dilution of step (a) that provides the preselected amount of reporter transgene expression relative to MAX or MAX−MIN in the presence of empty capsid AAV particles and/or HQC EV/HQC, storing, with the processor, HQC and/or HQC EV AND/OR HQC EV/HQC in the non-transitory electronic storage, and optionally outputting, with the processor, an indication of HQC and/or HQC EV and/or HQC EV/HQC for display.

### DESCRIPTION OF DRAWINGS

FIG. 1A shows 27% of healthy donor (HD; normal people without any known disease) samples had enhancers (E) which lead to incorrect estimates of AAV NAb titers in these samples. Also shown are 49% of HD have >1:40 AAV NAb titers, 14% of HD have 1:1 to 1:20 AAV NAb titers, and 10% of HD are negative for AAV NAb.

FIG. 1B shows that in the presence of enhancers, calculating AAV NAb titers is inaccurate. “BAb”=AAV binding antibody.

FIG. 2 shows detection of AAV NAb in serum using empty capsid AAV particles (EV).

FIGS. 3A-3E show outcomes of the 2-day assay using pre-incubation with empty capsid AAV particles (EV). X-axis titers are 1:1, 1:2.5, 1:5, 1:10, 1:100, and 1:1000. A) NAb⁺ (1:10); B) NAb⁻ (<1:1); C) NAb⁻ (<1:1), enhancers; D) NAb⁻ (<1:1), inhibitors, false positive; E) NAb⁺ (1:1), enhancers, false negative.

FIG. 4 shows decision tree for AAV NAb titer calculation.

FIGS. 5A and 5B shows representative formulas for calculating A) % Inhibition (MAX) and B) % Inhibition (SEV). MIN=Background.

FIGS. 6A-6E show examples of AAV NAb titer calculations. A) Decision tree for assigning AAV NAb titer according to test sample dilution; B) −sample; C) low NAb titer; D) enhancers in sample; E) high NAb titer.

FIG. 7 shows an exemplary assay plate layout.

FIGS. 8A-8C show assay criteria (1). A) Signal to noise ratio (S/N); B) EV efficacy; C) EV interference.

FIGS. 9A-9C show assay criteria (2).

FIG. 10A shows an exemplary FACT dilution scheme (wells 3B-3D).

FIG. 10B shows an exemplary samples dilution scheme (wells 4B-4G).

FIG. 11 shows exemplary transfer of diluted samples to Neutralization plate

FIG. 12 shows exemplary addition of FBS to Neutralization plate (Wells: MIN=2B; S=4B-4G; S.EV=5B-5G; FACT=3B-3D; FACT.EV=3E, 3F; MAX=2G; and MAX.EV=3G).

FIG. 13A shows exemplary addition of empty capsid AAV particles (EV) to Neutralization plate (Wells: MIN=2B; S=4B-4G; S.EV=5B-5G; FACT=3B-3D; FACT.EV=3E, 3F; MAX=2G; and MAX.EV=3G).

FIG. 13B shows exemplary addition of DMEM medium to Neutralization plate (Wells: MIN=2B; S=4B-4G; S.EV=5B-5G; FACT=3B-3D; FACT.EV=3E, 3F; MAX=2G; and MAX.EV=3G).

FIG. 14 shows exemplary addition of AAV reporter vector to Neutralization plate (Wells: MIN=2B; S=4B-4G; S.EV=5B-5G; FACT=3B-3D; FACT.EV=3E, 3F; MAX=2G; and MAX.EV=3G).

FIG. 15 shows controls and samples being transferred from the Neutralization to the Transduction plate (Wells: MIN=2B-2D; S=2E-2G; S.EV=9B-9G, 10B-10G and 11B-11G; FACT=3B-3D, 4B-4D and 5B-5D; FACT.EV=3E, 3F, 4E, 4F, 5E and 5F; MAX=2E-2G; and MAX.EV=3G, 4G and 5G).

FIG. 16 shows a schematic of the CAG promoter sequence (SEQ ID NO:3), composed of the cytomegalovirus (CMV) early enhancer element (“C”), first exon and first intron of the chicken beta-actin gene (“A”), and splice acceptor of the rabbit beta-globin gene (“G”).

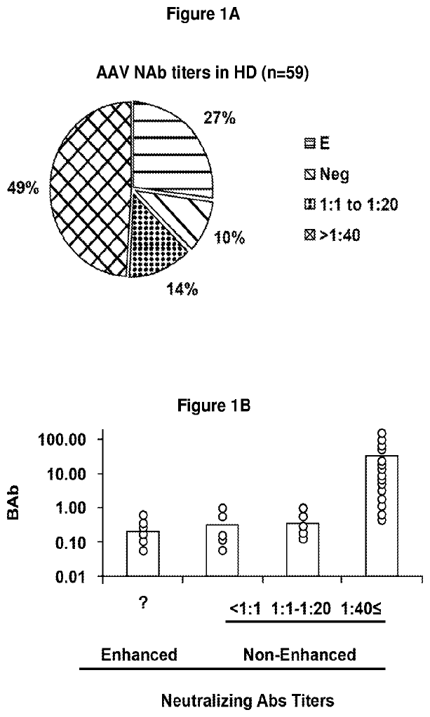

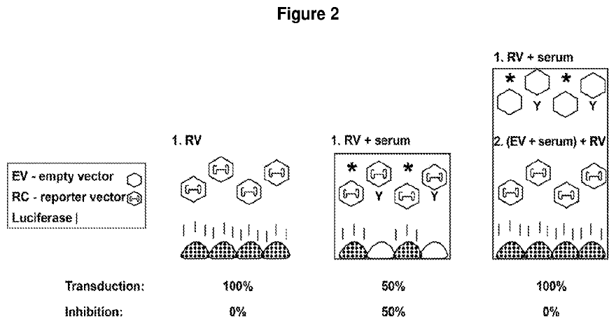

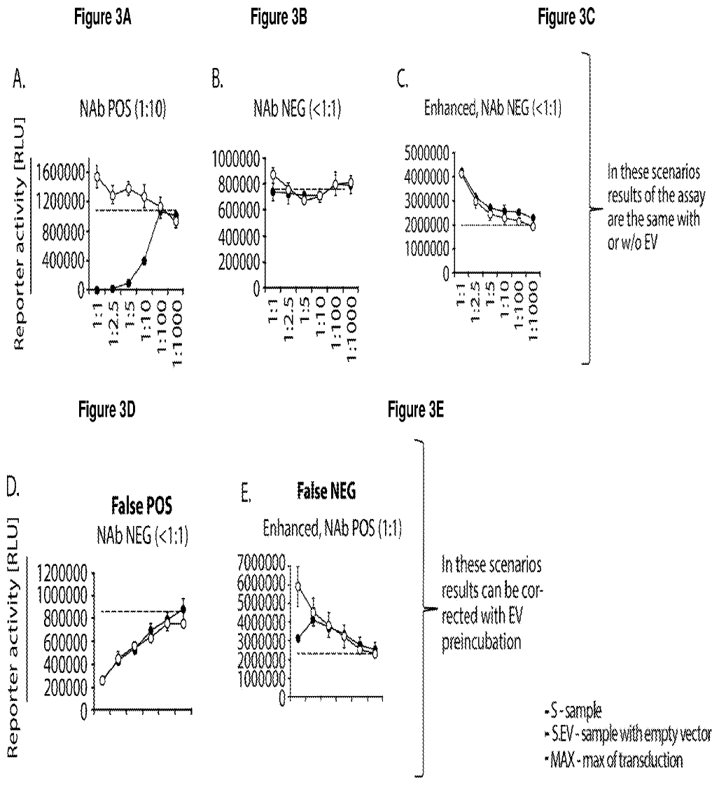

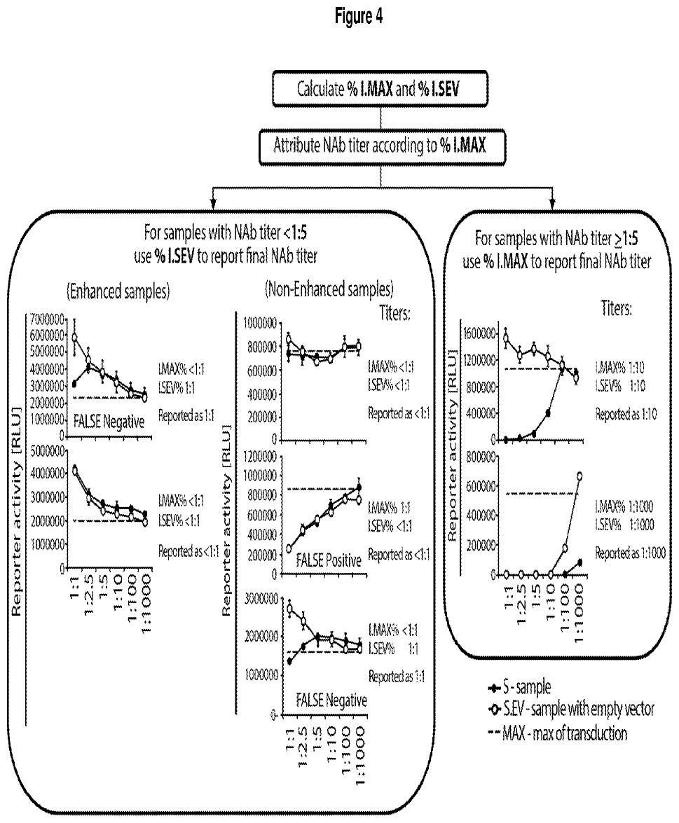

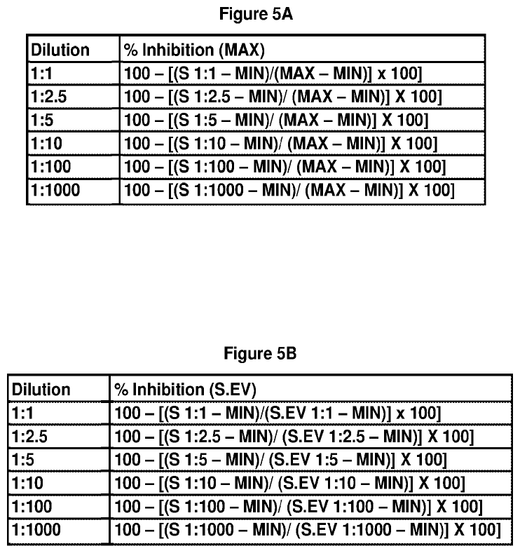

### DETAILED DESCRIPTION

Disclosed herein are methods for the detection of enhancers and inhibitors of AAV vector transduction and methods for detecting and/or quantifying AAV binding antibodies that inhibit, reduce or decrease AAV vector cell transduction, otherwise known as neutralizing antibodies (NAbs). Methods according to the invention rely, in part, on transduction of an AAV-permissive cell line with a reporter vector (an AAV particle carrying a reporter transgene). Methods according to the invention also rely, in part, on the use of empty capsid AAV particles to absorb a majority of or all AAV binding antibodies in a sample from a subject thereby revealing the presence of enhancers or inhibitors, if any, of AAV vector cell transduction in samples analyzed for AAV NAbs. Methods according to the invention may be used, inter alia, to more accurately determine AAV NAb titer in a sample from a subject, where the presence of enhancers or inhibitors can lead to false negatives and false positives, respectively.

In certain embodiments, certain steps in the assay methods according to the invention are optimized. In certain embodiments, the time of assay completion is reduced. In certain embodiments, assay variability is reduced and/or matrix interference is reduced. In particular, for example, compared to a conventional NAb assay (Meliani et al., 2015, Human Gene Therapy Methods, 26:45-53, doi: 10.1089/hgtb.2015.037) the assay methods according to the invention can be performed within about 2 days instead of 3 days, the intra-assay variation, assessed by Coefficient of Variation (% CV) of triplicate measures, was reduced from 30.5% to 8.7%, and the inter-assay precision assessment of the assay revealed a % CV of 12.5% for the quality control sample.

Methods according to the invention provide a streamlined, reliable and more accurate assay can be used to detect or quantify AAV binding antibodies that inhibit, reduce or decrease AAV vector cell transduction (AAV neutralizing antibodies (NAbs)) in a variety of contexts. For example, in certain embodiments the assay can be used to analyze for, detect or quantitate AAV NAbs to support selecting subjects for or excluding subjects from gene therapy treatments. In certain embodiments, the assay can be used to analyze for, detect or quantitate AAV NAbs for selecting subjects for or excluding subjects from inclusion in gene therapy trials. In certain embodiments, the assay can be used to analyze for, detect or quantitate AAV NAbs for monitoring subjects for development of anti-AAV antibodies after receiving a gene therapy treatment. In certain embodiments, the assay can be used to analyze for, detect or quantitate AAV NAbs for monitoring AAV NAbs in subjects who may need to or have received a treatment to reduce the amount of AAV NAbs.

In certain embodiments, a method for analyzing for, detecting or quantifying AAV binding antibodies that inhibit, reduce or decrease AAV vector cell transduction in a biological sample from a subject.

In certain embodiments, a method for analyzing for, detecting or quantifying AAV neutralizing antibodies that inhibit, reduce or decrease AAV vector cell transduction in a biological sample from a subject.

Adeno-associated virus (AAV) vectors are viral vectors that infect, inter alia, primates, such as humans.

As used herein and without limitation, the term “recombinant,” as a modifier of a viral vector, such as a recombinant AAV (rAAV) vector, as well as a modifier of sequences such as recombinant polynucleotides and polypeptides, means that compositions have been manipulated (i.e., engineered) in a fashion that generally does not occur in nature. A particular example of a recombinant AAV vector would be where a nucleic acid that is not normally present in a wild-type AAV genome (heterologous polynucleotide) is inserted within a viral genome. An example of which would be where a nucleic acid (e.g., gene) encoding a reporter transgene is cloned into a vector. Although the term “recombinant” is not always used herein in reference to an AAV vector, as well as sequences such as transgenes, recombinant forms including AAV vectors, polynucleotides, etc., are expressly included in spite of any such omission.

A “rAAV vector,” for example, is derived from a wild type genome of AAV by using molecular methods to remove all or a part of a wild type AAV genome, and replacing with a non-native (heterologous) nucleic acid, such as a reporter transgene encoding a reporter protein. Typically, for a rAAV vector one or both inverted terminal repeat (ITR) sequences of AAV genome are retained. A rAAV is distinguished from an AAV genome since all or a part of an AAV genome has been replaced with a non-native sequence with respect to the AAV genomic nucleic acid, such as with a reporter transgene encoding a reporter protein. Incorporation of a non-native (heterologous) sequence therefore defines an AAV as a “recombinant” AAV vector, which can be referred to as a “rAAV vector.”

A recombinant AAV vector sequence can be packaged, referred to herein as a “particle,” for subsequent infection (transduction) of a cell, ex vivo, in vitro or in vivo. Where a recombinant vector sequence is encapsidated or packaged into an AAV particle, the particle can also be referred to as a “rAAV,” “rAAV particle” and/or “rAAV virion.” Such rAAV, rAAV particles and rAAV virions include proteins that encapsidate or package a vector genome. Particular examples include in the case of AAV, capsid proteins.

As used herein, “infectious recombinant AAV particle(s)” refers to packaged recombinant AAV vector sequences that can infect or transduce a cell, ex vivo, in vitro or in vivo. As used herein, “cell(s) that can be infected” refers to cells receptive to infection or transduction by infectious recombinant AAV particles. In certain embodiments, methods of the invention use cells that can be infected by infectious rAAV particles in vitro.

A “vector genome,” which may be abbreviated as “vg,” refers to the portion of the recombinant plasmid sequence that is ultimately packaged or encapsidated to form a rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant AAV vector production, but is not itself packaged or encapsidated into rAAV particles. Thus, a “vector genome” refers to the nucleic acid that is packaged or encapsidated by rAAV.

Empty capsid AAV particles (EV) or empty vector (EV) refer to AAV particles that lack a vector genome. The empty capsid AAV particles are useful for absorbing (binding to) AAV binding antibodies in order to analyze for or detect the presence of enhancers of AAV vector cell transduction or inhibitors of AAV vector cell transduction. The empty capsid AAV particles are also useful in quantifying AAV NAb titers at a relative low titer (e.g., less than about 1:5).

As used herein, the term “serotype” in reference to an AAV vector means a capsid that is serologically distinct from other AAV serotypes. Serologic distinctiveness is determined on the basis of lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes). An antibody to one AAV capsid serotype may cross-react with one or more other AAV capsid serotypes due to homology of capsid protein sequence or a similar or identical conformational epitope.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV vectors and empty capsid AAV particles include any viral strain or serotype. For example and without limitation, an AAV vector genome or particle (capsid, such as VP1, VP2 and/or VP3) can be based upon any AAV serotype, such as AAV-1, -2, -3, -4, -5, -6, -7, -8, -9, -10, -11, -12, -rh74, -rh10, AA3B or AAV-2i8, for example. Such vectors and empty capsid AAV particles can be based on the same strain or serotype (or subgroup or variant) or be different from each other. For example and without limitation, a rAAV vector genome or particle (capsid) based upon one serotype genome can be identical to one or more of the capsid proteins that package the vector. In addition, a rAAV vector genome can be based upon an AAV serotype genome distinct from one or more of the capsid proteins that package the vector genome, in which case at least one of the three capsid proteins could be a different AAV serotype, e.g., AAV1, AAV2, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2), or variant thereof, for example. More specifically, a rAAV2 vector genome can comprise AAV2 ITRs but capsids from a different serotype, such as AAV1, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2), or variant thereof, for example. Accordingly, rAAV vectors include gene/protein sequences identical to gene/protein sequences characteristic for a particular serotype, as well as “mixed” serotypes, which also can be referred to as “pseudotypes.”

In certain embodiments, a AAV vector comprising the reporter has a capsid serotype identical to the capsid serotype of the empty capsid AAV particles. However, as long as empty capsid AAV particles are able to absorb (bind to) antibodies that bind to AAV vector comprising the reporter, due to cross-reactivity for example, the capsid serotype of the empty capsid AAV particles need not be the same serotype as the capsid serotype of the AAV vector comprising the reporter.

In certain embodiments, a rAAV vector or empty capsid AAV particles includes or consists of a capsid sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2) capsid proteins (VP1, VP2, and/or VP3 sequences). In certain embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, AAV12, -rh74, or -rh10 ITR(s).

In certain embodiments, rAAV vectors or empty capsid AAV particles include AAV1, AAV2, AAV3, AAV4, AAV5, AAV6, AAV7, AAV8, AAV9, AAV10, AAV11, AAV12, Rh10, Rh74, AAV3B and AAV-2i8 variants (e.g., capsid variants, such as amino acid insertions, additions, substitutions and deletions and ITR nucleotide insertions, additions, substitutions and deletions in the context of a rAAV vector) thereof, for example, as set forth in WO 2013/158879 (International Application PCT/US2013/037170), WO 2015/013313 (International Application PCT/US2014/047670) and US 2013/0059732 (U.S. application Ser. No. 13/594,773).

rAAV and empty capsid AAV particles, such as AAV1, AAV2, AAV3, AAV4, AAV5, AAV6, AAV7, AAV8, AAV9, AAV10, AAV11, AAV12, -rh74, -rh10, AAV3B, AAV-2i8, SPK1 (SEQ ID NO:1), SPK2 (SEQ ID NO:2) and variants, hybrids and chimeric sequences, can be constructed using recombinant techniques that are known to a skilled artisan, to include one or more heterologous polynucleotide sequences (transgenes) flanked with one or more functional AAV ITR sequences at the 5′ and/or 3′ end. rAAV vectors typically retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences).

In certain embodiments, an AAV vector is used to transduce target cells with a reporter transgene, which transgene is subsequently transcribed and optionally translated thereby providing a detectable signal to detect or quantitate transgene expression. The amount of signal is proportional to the efficiency of cell transduction and subsequent expression. Antibodies that bind to vector proteins that package or encapsidate the reporter transgene or inhibitor factors will inhibit, reduce or decrease vector cell transduction, subsequent reporter expression and therefore the amount of detectable signal.

In the assays described herein, for analyzing for, detection, and quantitation of antibodies selection of a particular capsid protein(s) serotype that packages or encapsidates the reporter transgene can be used to identify the serotype(s) the NAbs bind. For example, if it is desired to detect AAV-2 antibodies, the reporter transgene can be encpasidated by AAV-2 capsid protein(s). If it is desired to detect AAV-8 antibodies, the reporter transgene can be encapsidated by AAV-8 capsid protein(s). If it is desired to detect AAV-9 antibodies, the reporter transgene can be encpasidated by AAV-9 capsid protein(s). If antibody is present, the antibody binds to the capsid protein(s) that encapsidates the reporter transgene, inhibiting, reducing or decreasing vector cell transduction and consequent reporter transgene expression. The greater the quantity or titer of antibody that binds to envelope or capsid protein(s) the less vector transduction of cells and consequent reporter transgene expression and signal. Thus, the methods herein for analysis for, detection of, and quantifying antibodies that bind to vector proteins, such as viral (e.g., AAV) capsid proteins can also be used to identify the presence or absence of antibodies that bind to any particular AAV capsid serotype.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA).

Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

A “heterologous” transgene or nucleic acid refers to a polynucleotide inserted into a vector (e.g., AAV) for purposes of vector mediated transfer/delivery of the polynucleotide into a cell. Heterologous transgenes are distinct from vector (e.g., AAV) nucleic acid, i.e., are non-native with respect to viral (e.g., AAV) nucleic acid sequences. Once transferred/delivered into the cell, a heterologous transgene, contained within the virion, can be expressed (e.g., transcribed, and translated if appropriate). Although the term “heterologous” is not always used herein in reference to transgenes or nucleic acids, reference to a transgene or nucleic acid even in the absence of the modifier “heterologous” is intended to include heterologous transgenes and nucleic acids in spite of the omission.

As used herein, a “reporter” transgene is a polynucleotide that provides a detectable signal. The detectable signal may be provided by the reporter transgene itself, a transcript of the transgene or a protein encoded by the reporter transgene.

All mammalian and non-mammalian forms of transgenes including the examples without limitation of reporter transgenes and encoded proteins disclosed herein are expressly included, either known or unknown. Thus, the methods according to the invention includes reporter transgenes and proteins from microorganisms, and other organisms, which reporter transgenes and proteins are detectable in cells after transduction or transfer as described herein.

In certain embodiments, reporter transgenes encode a secreted or secretable protein. In certain embodiments, reporter transgenes encode a protein that provides an enzymatic, colorimetric, fluorescent, luminescent, chemiluminescent, or electrochemical signal.

For example and without limitation, reporter transgenes include luciferase genes which encode luciferase proteins, for example and without limitation, a Renilla luciferase, a firefly luciferase, or a Gaussia luciferase gene.

For example and without limitation, reporter transgenes may encode β-galactosidase, β-glucoronidase, chloramphenicol transferase, green fluorescent protein (GFP), red fluorescent protein (RFP) and alkaline phosphatase.

The “polypeptides,” “proteins” and “peptides” encoded by the “transgene” include full-length native sequences, as with naturally occurring proteins, as well as functional subsequences, modified forms or sequence variants, so long as the subsequence, modified form or variant retains some degree of functionality of the native full-length protein. In the invention, such polypeptides, proteins and peptides encoded by the transgene can be but are not required to be identical to the wild type protein.

In certain embodiments, a reporter transgene (the transgene provides a detectable signal) can comprise a single-stranded or a self-complementary genome. A self-complementary transgene becomes double stranded or is a double stranded dimer, when packaged into a virus particle (e.g., AAV vector) or upon virus vector cell transduction and virus uncoating within the transduced cell.

The terms “complementary” or “complement” when used in reference to a nucleic acid, such as a transgene, refers to a plurality of chemical bases such that through base pairing one single stranded sequence does or is capable of “specifically hybridizing” or binding (annealing) to another single stranded sequence to form a double-strand or duplex molecule. The ability of two single stranded sequences to specifically hybridize or bind (anneal) to each other and form a double-stranded (or duplex) molecule is by virtue of the functional group of a base on one strand (e.g., sense), which will hydrogen bond to another base on an opposing nucleic acid strand (e.g., anti-sense). The complementary bases that are able to bind to each other typically are, in DNA, A with T and C with G, and, in RNA, C with G, and U with A. Thus, an example of a self complementary sequence could be ATCGXXXCGAT, the X represents non-complementary bases, and the structure of such a double-stranded or duplex molecule with the X bases not hybridizing would appear as:

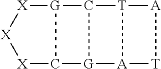

The terms “complementary” and “complement” when used in reference to a polynucleotide or nucleic acid, such as a transgene, is therefore intended to describe a physical state in which a double-stranded or duplex polynucleotide or nucleic acid molecule forms, or simply describes a sequence relationship between two polynucleotide or nucleic acid molecules such that each single strand molecule could form a double strand with the other. “Complementary” and “complement” therefore refers to the relationship of bases of each polynucleotide or nucleic acid molecule strand, and not that the two-strands must exist as a double stranded (or duplex) configuration or physical state with each other in a duplex.

Typically, for viral vectors that package single stranded nucleic acid, such as AAV, the inverted terminal repeat (ITR) sequences participate in replication and form a hairpin loop, which contributes to self-priming that allows initiation and synthesis of the second DNA strand. After synthesis of the second DNA strand, an AAV ITR has a so-called terminal resolution site (TRS), such that the hairpin loop is cleaved into two single strands each with a 5′ and 3′ terminal repeat for virus packaging.

Use of a deleted, mutated, modified, or non-functional TRS in at least one ITR results in formation of a double strand duplex that is not cleaved at the TRS. For a self-complementary reporter transgene double-stranded duplex structure, there is typically an ITR with a deleted, mutated or variant TRS located between the two complementary strands. The non-cleavable or non-resolvable TRS allows for self-complementary reporter transgene double-stranded duplex structure formation since the double strand form is not cleaved. Either the non-resolvable ITR with deleted, mutated or variant TRS, or resolvable ITR, may be suitable for virus packaging. Resolvable AAV ITR need not be a wild-type ITR sequence as long as the ITR mediates a desired function, e.g., packaging, self priming, replication, etc.

The ITR and TRS sequences of various AAV serotypes that may be deleted, mutated, modified, or varied include any AAV serotype set forth herein or that would be known to the skilled artisan. For example and without limitation, ITR and TRS sequences of various AAV serotypes include AAV-1, -2, -3, -4, -5, -6, -7, -8, -9, -10, -11, -rh74, -rh10. Another example is a modified or variant AAV ITR that is not processed by AAV Rep protein. Another example is a mutated, modified or variant AAV ITR with a deleted D sequence, and/or a mutated, modified or variant terminal resolution site (TRS) sequence. For AAV2, a representative mutated TRS sequence is: “CGGTTG.”

For self-complementary reporter transgene sequences positioned outside, such as one or more ITRs, expression regulatory sequences, downstream sequences, etc., such sequences of the vector sequence outside of the reporter transgene can, but need not be, self-complementary. Self-complementary can therefore be used in a specific context, for example, in reference to a transgene, such as a reporter transgene, such that only the transgene, such as the reporter transgene, is self-complementary, whereas the other non-transgene sequences may but need not be self-complementary.

To be self-complementary, not all bases in a single strand must be complementary to each and every base of the opposing complementary strand. There need only be a sufficient number of complementary nucleotide or nucleoside bases to enable the two polynucleotide or nucleic acid molecules to be able to specifically hybridize or bind (anneal) to each other. Hence, there may be short sequence segments or regions of non-complementary bases between the self-complementary polynucleotide or nucleic acid molecules. For example and without limitation, 1-5, 5-10, 10-20, 20-30, 30-40, 40-50, 50-75, 75-100, or 100-150 or more contiguous or non-contiguous non-complementary bases may be present but there may be sufficient complementary bases over the lengths of the two sequences such that the two polynucleotide or nucleic acid molecules are able to specifically hybridize or bind (anneal) to each other and form a double-strand (or duplex) sequence. Accordingly, sequences of the two single stranded regions may be less than 100% complementary to each other and yet still be able to form a double-strand duplex molecule. In certain embodiments, two single strand sequences have at least 70%, at least 75%, at least 80%, at least 85%, at least 90%, at least 95%, at least 98%, at least 99% or more complementarity to each other.

Such segments or regions of non-complementary bases between the self-complementary polynucleotide or nucleic acid molecules can be internal sequences, such that when the complementary portions of the two single stranded molecules form a double strand or duplex, the non-complementary bases form a loop or bulge configuration, and the overall structure resembles a hairpin. Such segments or regions of non-complementary bases between the self-complementary polynucleotide or nucleic acid molecules can also flank the complementary regions, in which case either or both if the 5′ or 3 flanking regions may not form a double-strand duplex.

In a cell having a transgene, the transgene has been introduced/transferred by way of vector, such as viral vector (e.g., AAV). This process is referred to as “transduction” or “transfection” of the cell. The terms “transduce” and “transfect” refers to introduction of a molecule such as a transgene into a cell.

A cell into which the transgene has been introduced is referred to as a “transduced or “transfected” cell. Accordingly, a “transduced,” or “transfected” cell, means a change in a cell following incorporation of an exogenous molecule, such as a polynucleotide (e.g., an AAV vector comprising a transgene) into the cell. Thus, a “transduced” or “transfected” cell is a cell into which, or a progeny thereof in which an exogenous molecule has been introduced, for example. The cell(s) can be propagated and the introduced transgene transcribed and/or protein expressed.

Cells that may be a target for transduction with a vector (e.g., viral vector) bearing transgene may be any cell susceptible to infection or that can be infected with the vector. Such cells include mammalian cells. Such cells may have low, moderate or high rates of susceptibility to infection. Accordingly, target cells include a cell of any tissue or organ type, of any origin (e.g., mesoderm, ectoderm or endoderm). Examples of cells that can be infected and that may be used in the methods according to the invention include, for example and without limitation, liver (e.g., hepatocytes, sinusoidal endothelial cells), pancreas (e.g., beta islet cells), lung, central or peripheral nervous system, such as brain (e.g., neural, glial or ependymal cells) or spine, kidney (HEK-293 cells), eye (e.g., retinal, cell components), spleen, skin, thymus, testes, lung, diaphragm, heart (cardiac), muscle or psoas, or gut (e.g., endocrine), adipose tissue (white, brown or beige), muscle (e.g., fibroblasts), synoviocytes, chondrocytes, osteoclasts, epithelial cells, endothelial cells, salivary gland cells, inner ear nervous cells or hematopoietic (e.g., blood or lymph) cells.

In certain embodiments, cells that may be a target for transduction with vector (e.g., viral vector) bearing transgene may be seeded from frozen cell aliquots or from a cell bank. In certain embodiments, cells that may be a target for transduction with vector (e.g., viral vector) bearing transgene may be seeded from cultured cells.

Examples of cell lines that can be a target for transduction with vector (e.g., viral vector) bearing transgene include, for example and without limitation, 2V6.11, HEK-293,CHO, BHK, MDCK, 10T1/2, WEHI cells, COS, BSC 1, BSC 40, BMT 10, VERO, WI38, MRCS, A549, HT1080, B-50, 3T3, NIH3T3, HepG2, Saos-2, Huh7, HER, HEK, HEL, and HeLa cells.

In certain embodiments, cells that may be a target for transduction with vector (e.g., viral vector) bearing transgene can transiently or stably express the E4 gene from adenovirus. E4 gene expression assures efficient cell transduction by AAV vectors.

AAV vectors and vector sequences can include one or more “expression control elements” or “expression regulatory elements.” Typically, expression control or regulatory elements are nucleic acid sequence(s) that influence expression of an operably linked polynucleotide (e.g. transgene). Control elements, including expression control and regulatory elements as set forth herein such as promoters and enhancers, present within a vector are included to facilitate proper transgene transcription and if appropriate translation (e.g., a promoter, enhancer, splicing signal for introns, maintenance of the correct reading frame of the polynucleotide to permit in-frame translation of mRNA and, stop codons etc.). Such elements typically act in cis but may also act in trans.

Expression control can be effected at the level of transcription, translation, splicing, message stability, etc. Typically, an expression control element that modulates transcription is juxtaposed near the 5′ end of the transcribed polynucleotide (i.e., “upstream”). Expression control elements can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, 5000 to 10,000 or more nucleotides from the polynucleotide), even at considerable distances. Nevertheless, owing to the polynucleotide length limitations, for AAV vectors, such expression control elements will typically be within 1 to 1000 nucleotides from the polynucleotide.

Functionally, expression of operably linked transgene is at least in part controllable by the element such that the element modulates transcription of the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence.

A “promoter” as used herein can refer to a DNA sequence that is typically located adjacent to a transgene. A promoter typically increases an amount expressed from a transgene compared to an amount expressed when no promoter exists.

An “enhancer” as used herein can refer to a sequence that is located adjacent to the transgene. Enhancer elements are typically located upstream of a promoter element but also function and can be located downstream of or within a DNA sequence (e.g., a transgene). Hence, an enhancer element can be located 100 base pairs, 200 base pairs, or 300 or more base pairs upstream or downstream of a transgene Enhancer elements typically increase expression of a transgene above increased expression afforded by a promoter element.

Examples of expression regulatory elements or expression control elements that can be used in methods according to the invention, include, for example and without limitation, CAG (SEQ ID NO:3), cytomegalovirus (CMV) immediate early promoter/enhancer, Rous sarcoma virus (RSV) promoter/enhancer, SV40 promoter, dihydrofolate reductase (DHFR) promoter, chicken (3-actin (CBA) promoter, phosphoglycerol kinase (PGK) promoter, and elongation factor-1 alpha (EF1-alpha) promoter.

Antibodies that bind to a recombinant viral vector useful for gene therapy, such as a rAAV vector, which can be referred to as “neutralizing” antibodies, can reduce, inhibit or decrease cell transduction by the viral vector. As a result, while not being bound by theory, cell transduction is reduced, inhibited or decreased, thereby reducing introduction of the viral packaged heterologous polynucleotide into cells and subsequent expression and, as appropriate, subsequent translation into a protein or peptide.

An immune response, such as hummoral immunity, can develop against a wildtype virus in a subject exposed to the wildtype virus. Such exposure can lead to pre-existing antibodies in the subject that bind to a viral vector based upon the wildtype virus, even prior to treatment with a gene therapy method employing the viral vector. Alternatively, antibodies may develop in a subject after treatment with a recombinant viral vector or after exposure to wildtype virus.

A biological sample is typically obtained from or produced by a biological organism. Examples of biological samples from a subject that may be analyzed using methods according to the invention include, for example and without limitation, whole blood, serum, plasma, the like, and a combination thereof. Other biological samples from a subject that may be used in methods according to the invention include, for example and without limitation, cerebrospinal fluid or simply spinal fluid. A biological sample may be devoid of cells, or may include cells (e.g., red blood cells, platelets and/or lymphocytes).

Suitable subjects from which a biological sample can be obtained for analysis by use of methods according to the invention include mammals, such as primates (e.g., humans), as well as non-human mammals The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Suitable human subjects include fetal, neonatal, infant, juvenile and adult subjects. Subjects also include animal disease models, for example, mouse and other animal models, such as nonhuman primates.

Suitable subjects (e.g., humans) from which a biological sample may be analyzed using methods according to the invention also include, for example and without limitation, those having loss-of-function and gain-of-function genetic diseases, disorders and defects. Accordingly, subjects (e.g., humans) include those that are candidates for or are undergoing gene replacement or supplement therapy, such as protein/enzyme replacement therapy, as well subjects (e.g., humans) that are candidates for or are undergoing gene knockdown or knockout therapy.

The term “loss-of-function” in reference to a genetic defect as used herein, refers to any mutation in a gene in which the protein encoded by the gene (i.e., the mutant protein) exhibits either a partial or a full loss of function that is normally associated with the wild-type protein. This includes any disease, disorder or defect caused by or resulting from insufficient expression or activity of a protein.

The term “gain-of-function” in reference to a genetic defect as used herein, refers to any mutation in a gene in which the protein encoded by the gene (i.e., the mutant protein) acquires a function not normally associated with the protein (i.e., the wild type protein) causes or contributes to a disease or disorder. The gain-of-function mutation can be a deletion, addition, or substitution of a nucleotide or nucleotides in the gene can give rise to the change in the function of the encoded protein. A gain-of-function mutation can change the function of the mutant protein or causes interactions with other proteins. A gain-of-function mutation can also cause a decrease in or removal of normal wild-type protein, for example, by interaction of the altered, mutant protein with the normal, wild-type protein. A gain of function mutation can lead to a disorder or disease caused by or resulting from expression or activity of an abnormal, aberrant or undesirable protein.

Suitable human subjects from which a biological sample may be analyzed using methods according to the invention include, for example and without limitation, subjects having a heritable disease or genetic disorder amenable to treatment by gene therapy. A gene therapy treatment or therapy includes vector (e.g., a viral vector such as AAV vector) mediated delivery of a nucleic acid for treatment of disease or disorder.

Suitable human subjects from which a biological sample may be analyzed using methods according to the invention further include, for example and without limitation, subjects having a lung disease (e.g., cystic fibrosis), a bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, a lysosomal storage disease (e.g., aspartylglucosaminuria, Batten disease, late infantile neuronal ceroid lipofuscinosis type 2 (CLN2), cystinosis, Fabry disease, Gaucher disease types I, II, and III, glycogen storage disease II, Pompe disease (caused by mutations in or loss of acid a-glucosidase (GAA; catalyzes the degradation of glycogen) function or expression), GM2-gangliosidosis type I (Tay Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A,B,C,D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), a disease of solid organs (e.g., brain, liver, kidney, heart), or an infectious viral (e.g., hepatitis B and C, HIV, etc.), bacterial or fungal disease. Suitable human subjects from which a biological sample may be analyzed according to methods of the invention additionally include subjects having blood clotting disorders, for example and without limitation, subjects having hemophilia A, hemophilia B, a deficiency in any coagulation Factor: VII, VIII, IX, X, XI, V, XII, II, von Willebrand factor, or a combined FV/FVIII deficiency, thalassemia, vitamin K epoxide reductase C1 deficiency or gamma-carboxylase deficiency.

Subjects from which a biological sample may be analyzed using methods according to the invention further include, for example and without limitation, those that have developed inhibitory antibodies against a protein delivered to the subject for therapeutic purposes, for example and without limitation, subjects having Pompe disease, hemophilia A, or hemophilia B administered GAA, Factor VIII, and Factor IX, respectively, can develop inhibitory antibodies against GAA, Factor VIII, and Factor IX, respectively. Accordingly, subjects include those not having inhibitory antibodies as well as subjects having inhibitory antibodies against a protein.

Suitable human subjects from which a biological sample may be analyzed according to methods of the invention moreover include subjects having a disease that affects or originates in the central nervous system (CNS) or a neurodegenerative disease, such as, for example and without limitation, Alzheimer's disease, Huntington's disease, ALS, hereditary spastic hemiplegia, primary lateral sclerosis, spinal muscular atrophy, Kennedy's disease, a polyglutamine repeat disease, Parkinson's disease, and polyglutamine repeat disease, inducing, for example and without limitation, spinocerebellar ataxias (e.g., SCA1, SCA2, SCA3, SCA6, SCAT, or SCA17).

The invention provides compositions, such as kits, that include packaging material and one or more components therein. A kit typically includes a label or packaging insert including a description of the components or instructions for use in vitro, in vivo, or ex vivo, of the components therein. A kit can contain a collection of such components, e.g., recombinant vector (e.g., rAAV) vector, empty capsid AAV particles, and optionally one or more reagents suitable for performing a method of the invention.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, quantities. Labels or inserts can include information identifying manufacturer, lot numbers, manufacture location and date, expiration dates. Labels or inserts can include information identifying manufacturer information, lot numbers, and date. Labels or inserts can include information on how a kit component may be used. Labels or inserts can include instructions for using one or more of the kit components in a method or use of the invention.

Unless otherwise defined, all technical and scientific terms used herein have the same meaning as commonly understood by one of ordinary skill in the art to which the invention belongs. Although methods and materials similar or equivalent to those described herein can be used in the practice or testing of the invention, suitable methods and materials are described herein.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

The term “about” as used herein refers to a value within 10% of the underlying parameter (i.e., plus or minus 10%). For example, “about 1:10” means 1.1:10.1 or 0.9:9.9, and about 5 hours means 4.5 hours or 5.5 hours, etc. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to 95% or more includes 95%, 96%, 97%, 98%, 99%, 100% etc., as well as 95.1%, 95.2%, 95.3%, 95.4%, 95.5%, etc., 96.1%, 96.2%, 96.3%, 96.4%, 96.5%, etc., and so forth. Thus, to also illustrate, reference to a numerical range, such as “1-4” includes 1, 2, 3, as well as 1.1, 1.2, 1.3, 1.4, etc., and so forth. For example, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013, etc., as well as 9.5, 9.6, 9.7, 9.8, 9.9, etc., and so forth. For example, a range of about “0.01 to about 10” includes 0.011, 0.012, 0.013, 0.014, 0.015, etc., as well as 9.5, 9.6, 9.7, 9.8, 9.9, etc., and so forth.

Reference to an integer with more (greater) or less than includes any number greater or less than the reference number, respectively. Thus, for example, reference to more than 2 includes 2.1, 2.2, 3, 3.1, 3.2, 4, 4.1, 4.2, 5, 5.1, 5.2, etc., and so forth. Reference to “two or more” includes 2, 3, 4, 5, 6, 7, 8, 9, 10, 11, 12, 13, 14, 15, or more times.

Further, reference to a numerical range, such as “1 to 90” includes 1.1, 1.2, 1.3, 1.4, 1.5, etc., as well as 81, 82, 83, 84, 85, etc., and so forth. For example, “between about 1 minute to about 90 days” includes 1.0 minutes, 1.1 minutes, 1.2 minutes, 1.3 minutes, 1.4 minutes, 1.5 minutes, etc., as well as one day, 2 days, 3 days, 4 days, 5 days . . . 81 days, 82 days, 83 days, 84 days, 85 days, etc.

The invention is generally disclosed herein using affirmative language to describe the numerous embodiments of the invention. The invention also specifically includes embodiments in which particular subject matter is excluded, in full or in part, such as substances or materials, method steps and conditions, protocols, or procedures. For example, in certain embodiments of the invention, materials and/or method steps are excluded. Thus, even though the invention is generally not expressed herein in terms of what the invention does not include, aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### EXAMPLES

### Example 1

This is a description of an exemplary cell-based in vitro assay for determining the anti-AAV neutralizing antibody (NAb) titer from serum or plasma samples using an AAV vector expressing luciferase as a reporter transgene.

This description is applicable to determine AAV NAb titers in serum and plasma and other biological samples from human clinical trial subjects, and pre- or non-clinical studies, human candidates for gene therapy treatment methods, as well as monitoring subjects for AAV NAbs, such as subjects who have received a gene therapy treatment or subjects who are at risk or have developed AAV NAbs, and where it is desirable to determine the presence or amount of NAbs before and/or after a treatment, and optionally to reduce the amount of AAV NAbs.

The exemplary 2V6.11 cell line used (Mohammadi, et al. Nucl. Acids Res. 32:2652 (2004)) is a genetically modified Human Embryonic Kidney (HEK) 293 cell line that stably expresses the E4 gene from adenovirus. E4 gene expression assures efficient transduction by AAV vectors. Other cells are appropriate for use.

The serum sample serves as the source of potential AAV NAbs, which, if present, will decrease AAV reporter vector transduction of the 2V6.11 cells and reduce measured luciferase activity. Serum sample is diluted, for example and without limitation, prepared in a range of dilutions (e.g. 1:1, 1:2.5, 1:5, 1:10, 1:100 and 1:1000) and mixed with the reporter vector, so AAV NAbs (if present) bind to the AAV reporter vector capsid surface and neutralization takes place.

Some humans, and therefore their serum (or other biological sample), may contain other factors that do not bind the AAV capsid but can affect AAV vector cell transduction positively (increase cell transduction) or negatively (decrease cell transduction), referred to herein respectively as enhancers and inhibitors. Thus, in parallel, serial dilutions of serum sample are also preincubated with empty capsid AAV particles (EV) followed by the addition of an AAV reporter vector.

Serum preincubation with empty capsid AAV particles (EV) provides a means to analyze low titer (e.g., less than or equal to about 1:5, or <1:5), or negative NAb samples for the presence of enhancers or inhibitors, for example in subjects prior to administration of AAV vector. This enables the identification of false NAb positive subjects, due to the presence of inhibitors, and false NAb negative subjects, due to the presence of enhancers. False NAb positive subjects can be treated by way of AAV vector based gene therapy methods. Depending on the NAb titer it may be decided to enroll or exclude false NAb negative subjects from AAV vector based gene therapy trials or methods. Clinical samples taken from subjects two weeks after AAV vector infusion typically have high NAb titer (e.g., greater than or equal to about 1:5, or >1:5) and using empty capsid AAV particles (EV) is not necessary for NAb titer determination in these subjects.

Quality controls (QC) can also be used to verify the integrity and/or accuracy of the assay. For example, AAV NAb controls can confirm that the empty capsid AAV particles (EV) are absorbing/binding to the AAV NAbs in order for the assay to determine the presence of any enhancers or inhibitors in a biological sample. Such a quality control is referred to herein as EV efficacy.

The source of AAV NAbs can be provided via one or more samples from one or more subjects that express AAV NAbs. Typically, AAV NAbs are from pooled samples to ensure the presence of AAV NAbs in the control. However, AAV NAbs in any form, such as in a solution of PBS, can function as control as disclosed herein.

2V6.11 cells are first transduced in a 96-well plate with a prepared suspension of AAV reporter vector, followed by overnight incubation. Luciferase activity is read by a luminescence plate reader. After background (MIN) subtraction for the entire plate, sample results, run in triplicate wells, are compared to the maximum (MAX) signal generated from the positive control wells containing cells transduced with the AAV reporter vector alone without serum. The dilution of the serum sample at which the luciferase activity is inhibited by about 50% or more is reported as the AAV NAb titer for the subject providing the sample.

In the exemplary study, samples were collected from 89 human subjects prior to enrollment in a phase 1/II gene therapy trial for treatment of hemophilia A. The assay revealed that out of the 89 subjects evaluated, 58 (65.2%) subjects had an AAV NAb titer of <1:1, which is considered negative; 16 (18%) subjects had a titer ≥1:1 but equal to or lower than 1:10 and 15 (16.8%) subjects had a titer >1:10.

### Example 2

| Key Terms | Definition |
|---|---|
| AAV | Adeno-Associated Virus |
| BSC | Biological Safety Cabinet |
| CV | Coefficient of Variation |
| cDMEM | Complete DMEM |
| DMEM | Dulbecco's Modified Eagles's Medium |
| DPBS | Phosphate Buffered Saline, no calcium, no magnesium |
| EV | Empty vector (AAV empty capsid particles) |
| FBS | Fetal Bovine Serum |
| GLuc | Gaussia luciferase |
| hr | hour |
| FACT | Factor Assay Control Plasma |
| Min | Minute |
| NA | Not Applicable |
| NAb | Neutralizing Antibody |
| NEG | Negative Control |
| POS | Positive Control |
| RLU | Relative luminescence units |
| RV | Reporter vector (e.g., AAV-CAG-GLuc2; AAV-CAG- |
| | Luciferase) |
| RT | Room temperature (ambient) |
| SD | Standard Deviation |
| Vol | Volume |
| MIN (Minimum of transduction) | Control of a background luminescence produced by culture plate or |
| | autoluminescence of the Renilla luciferase substrate, coelenterazine. This is |
| | caused by nonenzymatic oxidation of the coelenterazine in solution. Assay wells |
| | indicated as MIN contain only cells and FBS, but no AAV reporter vector. |
| MAX (Maximum of | Control that estimates the maximal level of cell transduction that can be obtained |
| transduction) | with AAV reporter vector. Assay wells indicated as MAX contain cells, FBS and |
| | AAV reporter vector, but no sample serum. |
| MAX.EV | Maximum of transduction (MAX) control supplemented with the empty capsid |
| | AAV particles. These wells provide the control values for an eventual |
| | transduction inhibition caused by the addition of empty capsid AAV particles to |
| | the assay wells. |
| Enhancement or enhancers | Term used to describe matrix interference within the assay. Sample is defined as |
| | enhanced when its luciferase activity is higher than in the Maximum of |
| | transduction (MAX) control. |
| Sample (S) | Sample is incubated with the AAV reporter vector (RV). These wells allow to |
| | determine NAb titer in non-enhanced samples compared to MAX control. |
| S.EV | Sample is first preincubated with the empty vector (EV) and secondly with AAV |
| | reporter vector (RV). These wells allow to determine AAV NAb presence in |
| | enhanced samples compared to MAX.EV |
| FACT (QC) | FACT is Pooled Normal Plasma and serves as assay quality control (QC). This |
| | product typically includes plasma from 30 or more carefully screened normal |
| | human donors. It contains AAV NAb and thus, controls the constancy of the |
| | assay performance. The AAV NAb titer is lot dependent. Other controls with |
| | AAV NAbs can also be used. |
| FACT.EV (QC.EV) | FACT is incubated with the empty vector (EV). These wells control that the |
| | addition of EV effectively can bind NAbs in each experiment. |
| Coefficient of Variation | CV measures the precision of repeated measurements. The formula for |
| | calculation is as follows: % CV = (Standard Deviation/Mean) × 100% |
| HQC | High quality control sample, a dilution of FACT (or other control with AAV |
| | NAbs) that produces about 80% inhibition of the maximal amount of |
| | transduction, as reflected by reporter expression, achieved in an uninhibited |
| | control; in other words a % I.MAX of about 80%. |
| MQC | Middle Quality Control sample, a dilution of FACT (or other control with AAV |
| | NAbs) that produces about 40-50% inhibition of the maximal amount of |
| | transduction, as reflected by reporter expression, achieved in an uninhibited |
| | control; in other words a % I.MAX of about 40-50%. |
| LQC | Low Quality Control sample, a dilution of FACT (or other control with AAV |
| | NAbs) that produces about 10% inhibition of the maximal amount of |
| | transduction, as reflected by reporter expression, achieved in an uninhibited |
| | control; in other words a % I.MAX of about 10%. |

### Example 3

### Materials and Equipment

Cells were 2V6.11 cell line (Mohammadi, et al. Nucl. Acids Res. 32:2652 (2004)), which is a Human Embryonic Kidney (HEK) cell line stably expressing Adenoviral E4 gene. Master, Working, and Assay-Ready Cell Banks are generated. The “cell bank” aliquots, 1e7/mL, are stored in liquid nitrogen.

### Reagents and Storage Conditions

| | Storage |
|---|---|
| Reagents | Condition |
| Density-gradient purified AAV-CAG-GLuc2 vector. | −80° C. |
| AAV Empty capsid particles. | −80° C. |
| Renilla Luciferase Assay System, Promega, | −20° C. |
| Cat # E2820. | |
| FACT (Pooled Normal Plasma), George King | −80° C. |
| Bio-Medical INC Cat# 0020-1 | |
| Ponasterone A, Life Technologies, Cat # H101-01, | −20° C. |
| DMEM (Dulbecco's Modified Eagle's Medium), | 4° C. |
| Life Technologies, Cat # 11965-084 | |
| DPBS (Dulbecco's Phosphate Buffered Saline), | ambient |
| no calcium, no magnesium, Life Technologies, | |
| Cat # 14190-235 | |
| FBS, heat-inactivated, Certified, US origin, | −20° C. |
| Life Technologies Cat # 10082147 | |
| Penicillin-Streptomycin-Glutamine (100X), | −20° C. |
| Gibco, Cat #10378-016 | |
| Pluronic ™ F-68 Non-ionic Surfactant (100X, | ambient |
| 10%), ThermoFisher Scientific, Cat# 24040032 | |
| 100% ethanol, Sigma, Cat # E7023-500 ml | ambient |
| Deionized water from in-house Milli-Q Water | ambient |
| Purification System | |
| Trypan Blue, Invitrogen, Cat # T10282 | ambient |
| Trypsin-EDTA (0.25% trypsin with EDTA 4Na), | 4° C. |
| Life Technologies, Cat # 25200-05 (only | |
| for cell culture) | |

### Example 4

### Reagent Preparation

Reagent volume may be scaled as needed.

Add 50 mL of heat inactivated FBS to 55 mL of complete DMEM (cDMEM). Add 5 mL of 100× Pen/Strep/Glutamine solution. Filter with sterile bottle filter. Assign an expiration date of 1 month and store at 4° C.

FACT QC stock: The AAV NAb titers in various lots of FACT may differ and dilution scheme may be adapted in a way to achieve 50% inhibition between two intermediate dilutions. Other control AAV NAbs may be used, such as plasma or serum.

Example: For Lot 3696, dilute the heat-inactivated FACT sample at 1:10 with heat inactivated FBS and freeze in aliquots. Store at <−60° C. and assign expiration date. Do not exceed 3 freeze-thaw cycles. The HQC, MQC, and LQC are diluted during assay set up using the following exemplary scheme:

TABLE 1
Example of Dilution Scheme for QCs (Used for FACT Lot 3696)

| QC description | Vol of FACT | Vol of FBS |
|---|---|---|
| FACT 1:100 (HQC) | 10 parts of FACT 1:10 | 90 parts of FBS |
| FACT 1:316 (MQC) | 1 part of FACT 1:100 | 2.16 parts of FBS |
| FACT 1:1000 (LQC) | 1 part of FACT 1:316 | 2.16 parts of FBS |

Ponasterone A: Quick spin the vial and reconstitute in 100% ethanol at a concentration of 1 mg/mL. Vortex vigorously. Store at −20° C.

Pluronic F68 in DPBS: Mix one part of 10% Pluronic stock solution with 10,000 parts of DPBS using a two-tier dilution scheme. Use on the date of preparation.

AAV-CAG-Luciferase Vector: Upon initial thaw of the manufacture stock, dilute the vector with 0.001% Pluronic F68 to obtain stock concentration of 2×10¹¹ vg/mL. Distribute as 50 μL aliquots into sterile nonstick surface tubes. Store aliquots at −60° C.

Empty capsid AAV particles (EV): After initial thaw of manufacture stock, aliquot appropriate volumes into sterile nonstick surface tubes and store at −60° C. Discard the remainder after thawing the aliquot.

Test Samples: After the initial thawing, heat inactivate serum samples at 56° C. for 30 minutes. Prepare 400 μL aliquots in sterile non-stick surface tubes and store at −60° C. until use. Once thawed, unused serum can be saved and stored at −60° C. or can be discarded. If saved, mark freeze-thaw cycle on tube. Do not exceed 3 freeze-thaw cycles.

Diluent Serum: Fetal Bovine Serum (FBS) Prepare single-use aliquots, and store at −20° C.

1× Renilla Luciferase Assay Reagent: Thaw or bring to room temperature the assay buffer and substrate (ambient temperature). A water bath can be used. Mix well, as thawing generates density and composition gradients. Reagents may be thawed up to five times without appreciable activity loss. To prepare reagent add 1 volume of 100× Renilla Luciferase Assay Substrate to 100 volumes of Renilla Luciferase Assay Buffer in a 50 mL conical tube. Mix thoroughly by vortexing the tube 10-20 seconds. Keep reagent in the dark. Once prepared, the buffer is stable for 12 hours at ambient temperature.

### Example 5

Day 1: Plating 2V6.11 Cells, Sample Neutralization and Cell Transduction Place the cDMEM medium in a 37° C. CO₂ incubator with a loose cap for a minimum of 30 minutes. This allows the pH and the temperature to reset. This medium is used later for cell thawing and plating.

Remove samples, FACT QC stock, heat-inactivated FBS and DMEM without FBS from storage and have them equilibrated to ambient temperature.

Optional: If the serum sample has not been heat-inactivated, heat-inactivate the samples at 56° C. for 30 minutes. Label the tube to indicate heat-inactivation. Do not heat samples again if previously heat inactivated.

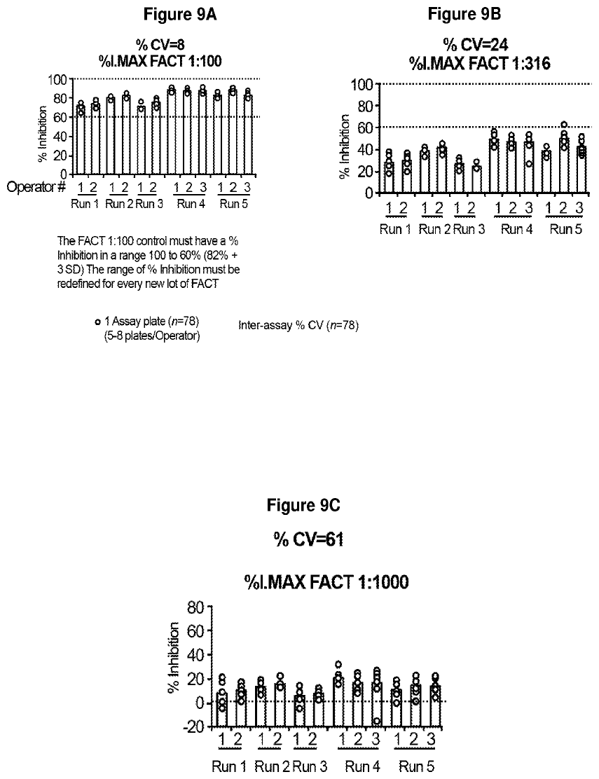

Use a 96-well U-bottom tissue culture plate to prepare the dilutions from FACT QC stock solution as shown in FIG. 10A. This plate is called a Dilution plate. Indicated volumes are enough for preparation of one sample and should be scaled up if needed. Pipette to mix until homogenous. Using the same dilution plate, prepare sample dilutions as indicated in FIG. 10B.

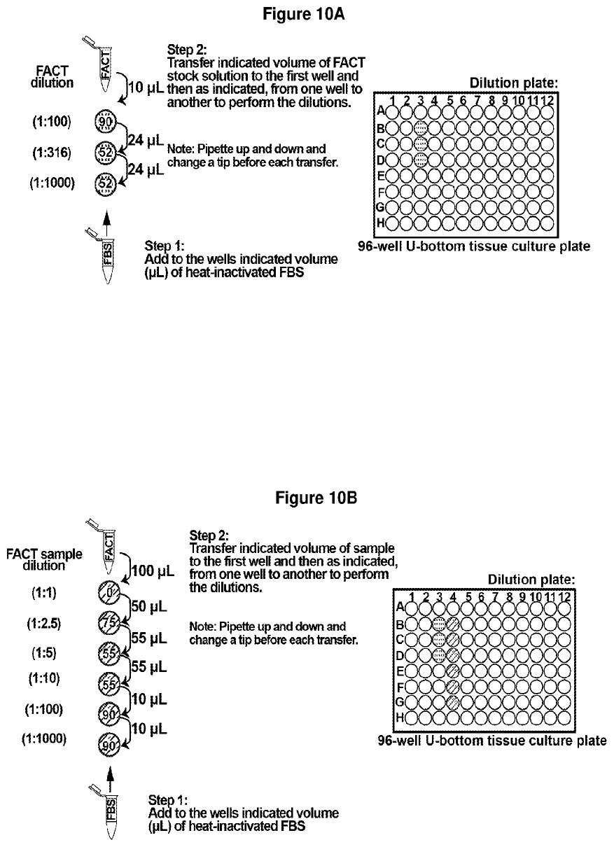

Transfer 20 μL of each diluted FACT control and the sample from the dilution plate to the new 96-well U-bottom Neutralization plate as shown in FIG. 11.

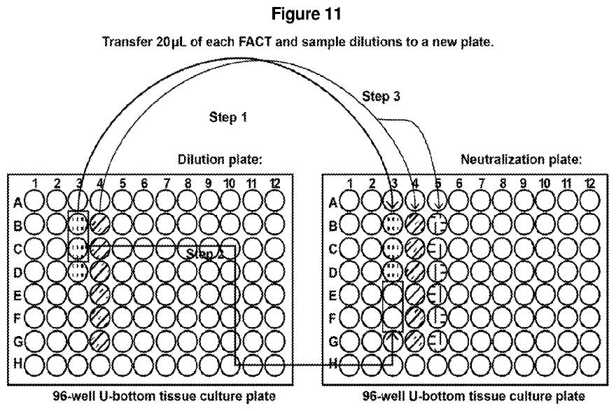

Add 20 μL of heat-inactivated FBS to the MIN, MAX and MAX.EV well on the Neutralization plate as shown in FIG. 12.

Only when EV is used, prepare a working concentration of the empty capsid AAV particles (EV) of 1.5×10¹¹ cp/mL in DMEM without FBS. Avoid vigorous vortexing. A volume of 0.8 mL of diluted EV is sufficient for one full assay plate.

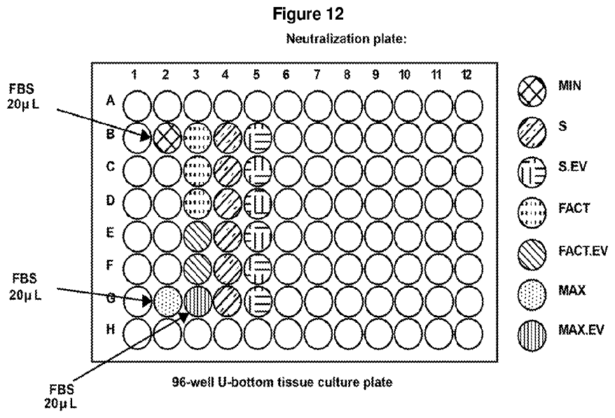

Only when EV is used, add 10 μL of the EV to the wells designated S.EV, FACT.EV and MAX.EV, as shown in FIG. 13A. Add 10 μL of DMEM without FBS to the wells containing samples (S), FACT and MAX, and 20 μL of DMEM without FBS to MIN control well as shown in FIG. 13B. Only when EV is used, incubate the Neutralization plate 30±5 minutes at 37° C.

Prepare a working concentration of the AAV reporter vector (RV) of 3.2×10⁹ vg/mL in DMEM without FBS. Avoid vigorous vortexing. A volume of 0.8 mL of diluted vector is sufficient for one full assay plate.

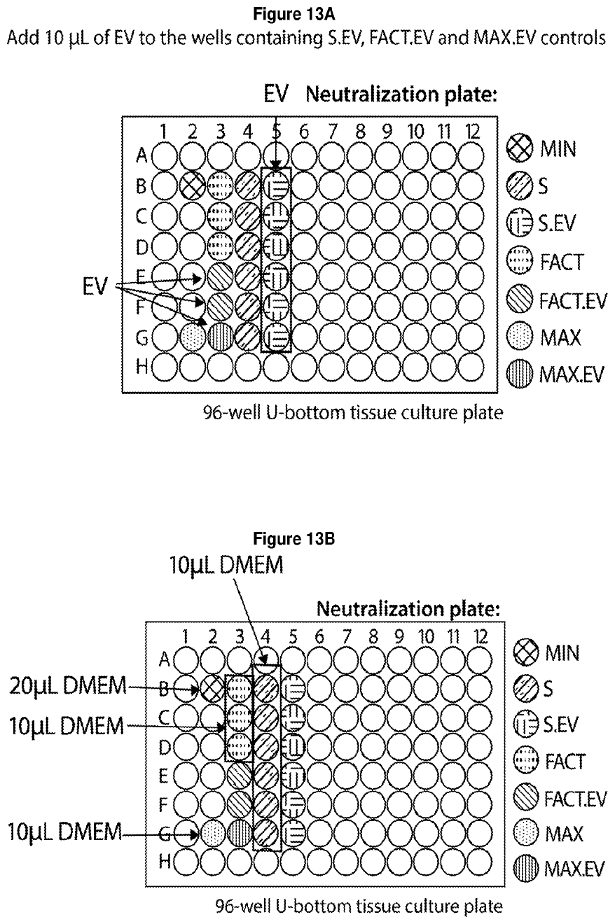

Add 10 μL of the RV to all wells, except the well containing MIN control in the Neutralization plate as shown in FIG. 14. Incubate the Neutralization plate for 30±5 minutes at 37° C.

Remove one vial of 2V6.11 cells from cryostorage and place immediately in 37° C. water bath for 4 minutes. If multiple vials are needed, scale up accordingly. Remove the vial from water bath, wash with 70% EtOH and flip it twice 180° to resuspend the cells.

Use 1 or 2 mL serological pipette to aspirate all cell suspension from the cryovial and transfer it into an empty 15 mL conical tube. Cells are fragile to shearing force at this step.

Using a 10 mL pipette, add 9 mL of warm medium to the 15 mL conical tube. The first 3 mL should be added slowly, drop-by-drop. Add the remaining 6 mL of medium more quickly from the pipette. The cells are now suspended in ˜10 mL.

Close the 15 mL conical tube and flip it twice 180° to homogenize the cell suspension. Transfer 50 μL of the cell suspension to an Eppendorf tube for counting purpose and spin the remaining cell suspension at 240×g for 10 minutes.

Count live and dead cells. If viability is lower than 70% discard the cells and thaw another vial.

Dilute the cells to 4.0×10⁵ cells/mL in cDMEM. Add ponasterone A to a final concentration of 1 μg/mL. Each plate needs 10 mL of cell suspension.

Take a flat-bottom 96-well tissue culture plate and add 100 μL of cell suspension (4.0×10⁴ cells) per well. This plate is called a Transduction plate. Incubate at 37° C., 5% CO₂ incubator until use. Document lot number of the cell bank used, and viability.

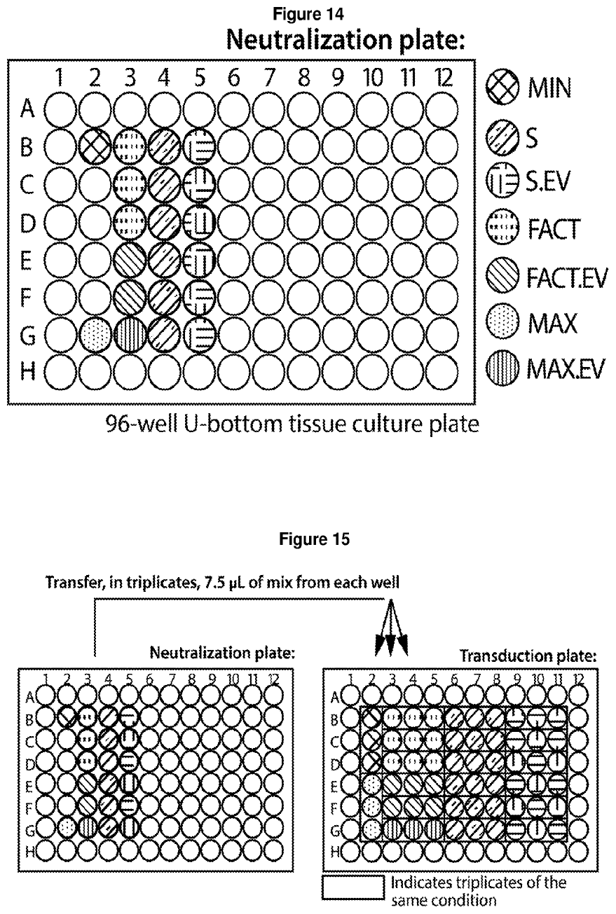

After the neutralization, transfer 7.5 μL from each well on the Neutralization plate to triplicates wells in the Transduction plate as shown in FIG. 15. Wrap the Transduction plate with parafilm and incubate the Transduction plate at 37° C., 5% CO₂ tissue culture incubator for 24 hours ±30 minutes. Record the incubation starting time.

### Day 2: Measurement of Luciferase Activity

Cell culture supernatant containing secreted Luciferase should be taken from the wells within about 24 to 25 hours after addition of the 7.5 μL/well transferred from the Neutralization plate to the Transduction plate. Since the secreted Luciferase accumulates in the culture medium with time, varying this time will greatly affect the range of activity readings.

Prepare sufficient volume of 1× Renilla Luciferase Assay Reagent and allow to equilibrate to room temperature prior to assay. Remove the assay plate from the incubator. Observe the cells and document any sign of toxicity (low confluency, cells detached from the well bottom, cells attached but round shape, etc.) and note.

Turn GloMax Navigator System microplate reader on and then turn tablet PC on for at least five minutes prior to reading. When the ˜24-hour incubation is over, pipet the supernatant up and down once with a multi-channel pipet, and transfer ˜90 μL of the culture supernatant to a 96-well tissue culture plate. The excess of supernatant may serve for second reading if desired. If not used, may be discarded or frozen <−60° C.

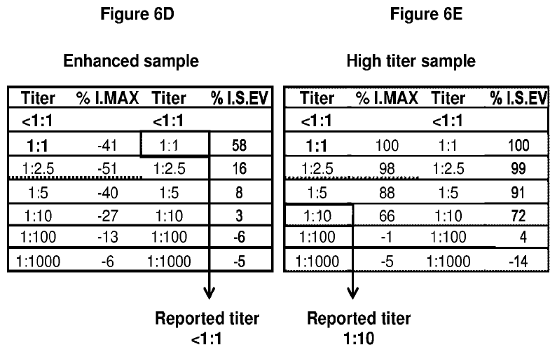

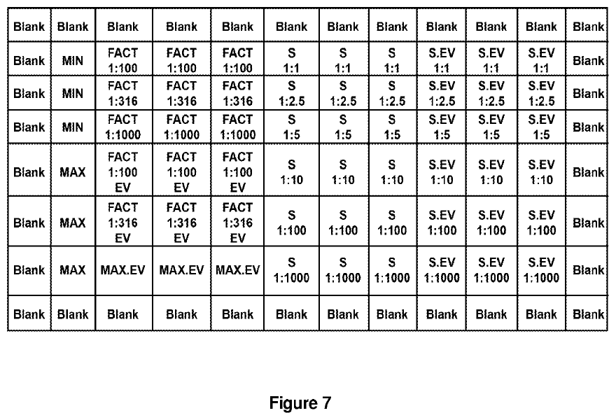

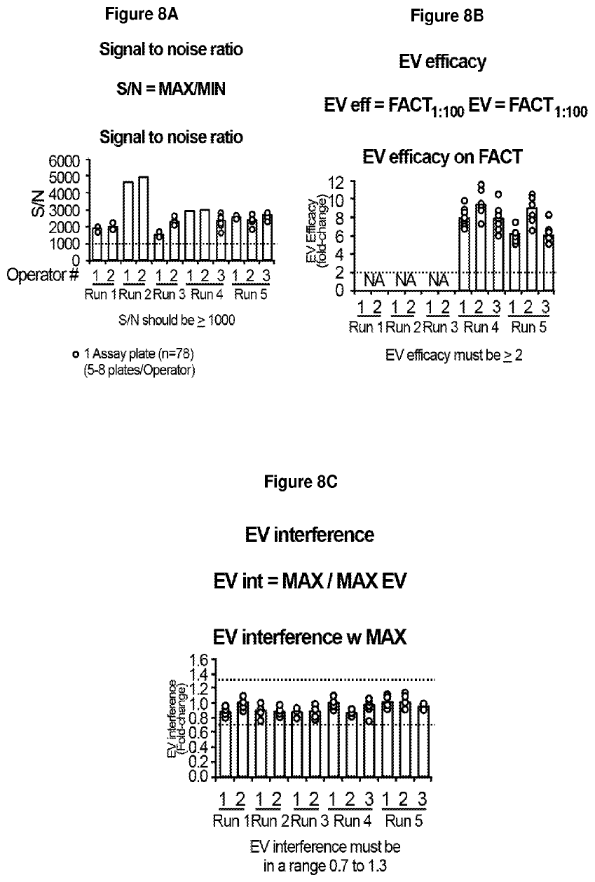

Transfer 40 μL of the culture supernatant to a 96-well black plate. The assay plate layout is shown in FIG. 7. Optionally, if the plate is not read immediately, seal the plate with a sealer and store it in a dark condition at room temperature for up to 4 hours.

Load the reader with the plate. Tap the GloMax Navigator Software Icon to launch the GloMax Navigator software. Select the protocols. Use the instrument settings detailed below:
Read Mode: Luminescence
Integration Time: 1 sec
Plate Type: 96-well Standard
Read Area: Highlight the columns and rows to be measured
Injection & Delay:
Injector 1: deselect
Injector 2: select
Volume: 100 μL
Delay: 2 Sec
Speed: 230 μL /Sec
Dark Adapt: 3 minutes
Read the plate

### Example 6

### Data Analysis

Calculate the average (also referred to as “mean”) standard deviation (SD), and % CV of luminescence readings [RLU] for all controls and sample dilutions.

Use the average luminescence values [RLU_{AV}] of MAX triplicate wells and MIN triplicate wells to calculate the signal-to-noise ratio (S/N). S/N=MAX[RLU_{AV}]/MIN[RLU_{AV}]

Use the average luminescence values [RLU_{AV}] of MAX, MIN, QC dilutions with and without empty vector (e.g., HQC, MQC, LQC, HQC EV, MQC EV and LQC EV), and the test sample (S) triplicate wells, respectively, for the following calculations:

% Inhibition MAX (%I.MAX)=100−[[(S−MIN)/(MAX−MIN)]×100]. This is calculated for each sample (S) dilution, as shown in Table 2.

TABLE 2

| Dilution | % Inhibition MAX (% I.MAX) |
|---|---|
| 1:1 | 100 − [(S 1:1 − MIN)/(MAX − MIN)] × 100] |
| 1:2.5 | 100 − [(S 1:2.5 − MIN)/(MAX − MIN)] × 100] |
| 1:5 | 100 − [(S 1:5 − MIN)/(MAX − MIN)] × 100] |
| 1:10 | 100 − [(S 1:10 − MIN)/(MAX − MIN)] × 100] |
| 1:100 | 100 − [(S 1:100 − MIN)/(MAX − MIN)] × 100] |
| 1:1000 | 100 − [(S 1:1000 − MIN)/(MAX − MIN)] × 100] |

% Inhibition S.EV (%I.SEV)=100−[[(S−MIN)/(S.EV−MIN)]×100]. This is calculated for each sample (S) dilution, as shown in Table 3.

TABLE 3

| Dilution | % Inhibition S.EV (% I.SEV) |
|---|---|
| 1:1 | 100 − [(S 1:1 − MIN)/(S.EV 1:1 − MIN)] × 100] |
| 1:2.5 | 100 − [(S 1:2.5 − MIN)/(S.EV 1:2.5 − MIN)] × 100] |
| 1:5 | 100 − [(S 1:5 − MIN)/(S.EV 1:5 − MIN)] × 100] |
| 1:10 | 100 − [(S 1:10 − MIN)/(S.EV 1:10 − MIN)] × 100] |
| 1:100 | 100 − [(S 1:100 − MIN)/(S.EV 1:100 − MIN)] × 100] |
| 1:1000 | 100 − [(S 1:1000 − MIN)/(S.EV 1:1000 − MIN)] × 100] |

HQC % Inhibition=100−[(HQC−MIN)×100)/(MAX−MIN)]

EV Interference=MAX/MAX.EV

EV Efficacy=HQC EV/HQC

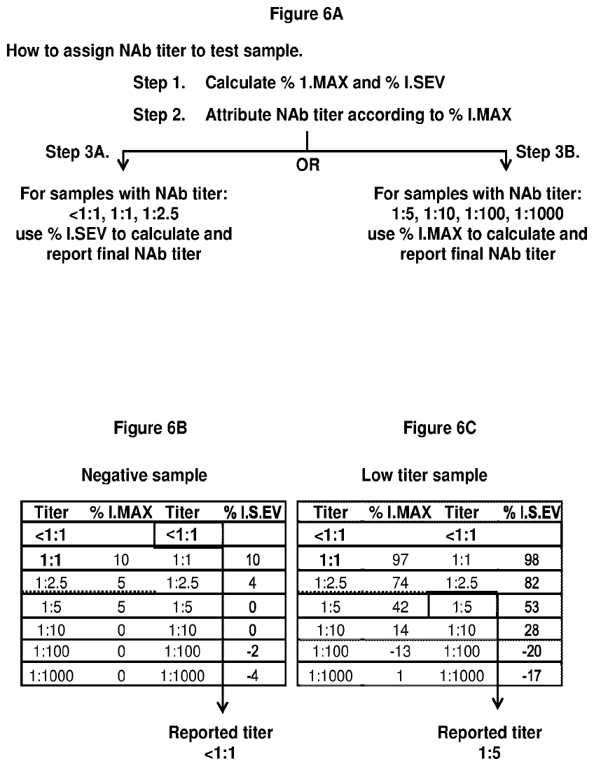

AAV NAb titer is defined as the lowest sample dilution with the % Inhibition >50. Assign NAb titer to each sample as shown in FIGS. 6A-6E.

### Assay Acceptance Criteria

Assay Criterion 1: The HQC (e.g., FACT 1:100) must meet pre-defined lot-specific % I.MAX±3 SD.

Assay Criterion 2: The HQC (e.g., FACT 1:100) must meet precision of CV %≤35% for values [RLU] among triplicate wells.

Assay Criterion 3: The positive transduction control (MAX) must meet precision of CV %≤35% for luminescence values [RLU] among triplicate wells.

Assay Criterion 4 (Applicable only when EV is used in the assay): The calculated EV interference must be in a range 0.7 to 1.3.

Assay Criterion 5 (Applicable only when EV is used in the assay): The calculated EV must be ≥2.

An assay plate that does not meet all the above applicable criteria is considered a failed plate. No result should be reported from a failed plate. The assay is repeated.

### Sample Titer Acceptance Criteria

Criterion 1: The sample dilution which is reported as a final NAb titer should meet precision of CV %≤35% for luminescence values [RLU] among triplicate wells. For any sample that does not meet the criteria, the result is considered invalid and a retest is warranted, unless the sample is depleted or specifically justified. Samples with valid results should not be re-analyzed, unless a technical error is identified, or re-analysis is desired.

### Example 7

TABLE 4

| | PD17-32 | R&D Assay | Improvement |
|---|---|---|---|
| Time | 3 days | 2 days | Shorter assay |
| Cells | Cells in culture from | Frozen cell aliquots | Lower lot-to-lot |
| | passage 3 to 16 | | variability with |
| | | | frozen aliquots |
| No. Cells/Well | 20,000 | 40,000 | Lower variability due |
| | (50% confluency) | (90% confluency) | to higher seeded cell |
| | | | number/confluency |
| Reporter Vector | Multiplicity of | MOI 150 | Decreased % CV |
| | infection (MOI) 15 | | with higher MOI |
| Reporter Gene | Intracellular | Secretable luciferase | Secretable eliminates |
| | luciferase | | lysis step |
| Reporter Gene | Renilla luciferase | Gaussia luciferase | Stronger signal |
| | MAX = 10,000 RLU | MAX = 1,000,000 | permits using black |
| | | RLU | plates |
| Luminometer | White plates | Black plates | Black plates |
| Plates | | | eliminate crosstalk |
| | | | between wells |

### Example 8

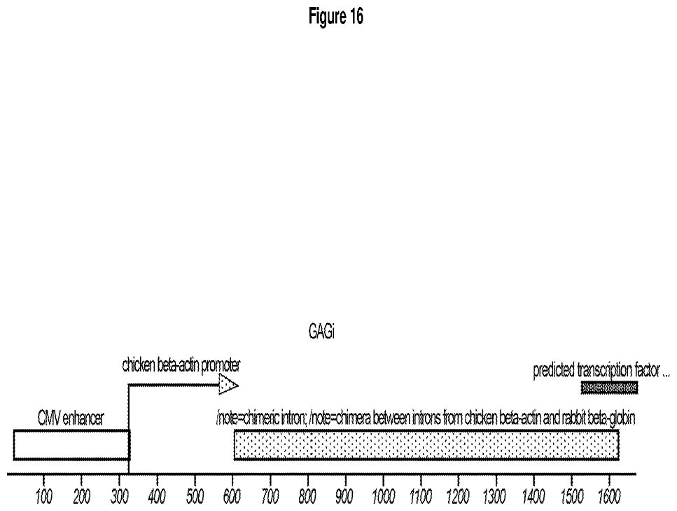

Spk1 (SEQ ID NO: 1):
MAADGYLPDWLEDNLSEGIREWWDLKPGAPKPKANQQKQDNGRGLVLPGY
KYLGPFNGLDKGEPVNAADAAALEHDKAYDQQLQAGDNPYLRYNHADAEF
QERLQEDTSFGGNLGRAVFQAKKRVLEPLGLVESPVKTAPGKKRPVEPSP
QRSPDSSTGIGKKGQQPAKKRLNFGQTGDSESVPDPQPIGEPPAAPSGVG
PNTMAAGGGAPMADNNEGADGVGSSSGNWHCDSTWLGDRVITTSTRTWAL
PTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYNFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFNQAKLASFITQYSTGQVSVEIEWELQKENSKRWNPE
IQYTSNYYKSTNVDFAVNTEGTYSEPRPIGTRYLTRNL
Spk2 (SEQ ID NO:2):
MAADGYLPDWLEDNLSEGIREWWALQPGAPKPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVDQSP
QEPDSSSGVGKSGKQPARKRLNFGQTGDSESVPDPQPLGEPPAAPTSLGS
NTMASGGGAPMADNNEGADGVGNSSGNWHCDSQWLGDRVITTSTRTWALP
TYNNHLYKQISSQSGASNDNHYFGYSTPWGYFDFNRFHCHFSPRDWQRLI
NNNWGFRPKKLSFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFQFSYTFEDVPFHSSYAHSQSLDRLMNPLIDQYLYYLNRTQG
TTSGTTNQSRLLFSQAGPQSMSLQARNWLPGPCYRQQRLSKTANDNNNSN
FPWTAASKYHLNGRDSLVNPGPAMASHKDDEEKFFPMHGNLIFGKEGTTA
SNAELDNVMITDEEEIRTTNPVATEQYGTVANNLQSSNTAPTTRTVNDQG
ALPGMVWQDRDVYLQGPIWAKIPHTDGHFHPSPLMGGFGLKHPPPQIMIK
NTPVPANPPTTFSPAKFASFITQYSTGQVSVEIEWELQKENSKRWNPEIQ
YTSNYNKSVNVDFTVDTNGVYSEPRPIGTRYLTRPL
CAG Promoter Sequence (also shown schematically
in FIG. 16) (SEQ ID NO: 3):
ATAGCCCATATATGGAGTTCCGCGTTACATAACTTACGGTAAATGGCCCG
CCTGGCTGACCGCCCAACGACCCCCGCCCATTGACGTCAATAATGACGTA
TGTTCCCATAGTAACGCCAATAGGGACTTTCCATTGACGTCAATGGGTGG
AGTATTTACGGTAAACTGCCCACTTGGCAGTACATCAAGTGTATCATATG
CCAAGTACGCCCCCTATTGACGTCAATGACGGTAAATGGCCCGCCTGGCA
TTATGCCCAGTACATGACCTTATGGGACTTTCCTACTTGGCAGTACATCT
ACGTATTAGTCATCGCTATTACCATGGTCGAGGTGAGCCCCACGTTCTGC
TTCACTCTCCCCATCTCCCCCCCCTCCCCACCCCCAATTTTGTATTTATT
TATTTTTTAATTATTTTGTGCAGCGATGGGGGCGGGGGGGGGGGGGGGGC
GCGCGCCAGGCGGGGCGGGGCGGGGCGAGGGGCGGGGCGGGGCGAGGCGG
AGAGGTGCGGCGGCAGCCAATCAGAGCGGCGCGCTCCGAAAGTTTCCTTT
TATGGCGAGGCGGCGGCGGCGGCGGCCCTATAAAAAGCGAAGCGCGCGGC
GGGCGGGGAGTCGCTGCGACGCTGCCTTCGCCCCGTGCCCCGCTCCGCCG
CCGCCTCGCGCCGCCCGCCCCGGCTCTGACTGACCGCGTTACTCCCACAG
GTGAGCGGGCGGGACGGCCCTTCTCCTCCGGGCTGTAATTAGCGCTTGGT
TTAATGACGGCTTGTTTCTTTTCTGTGGCTGCGTGAAAGCCTTGAGGGGC
TCCGGGAGGGCCCTTTGTGCGGGGGGAGCGGCTCGGGGGGTGCGTGCGTG
TGTGTGTGCGTGGGGAGCGCCGCGTGCGGCTCCGCGCTGCCCGGCGGCTG
TGAGCGCTGCGGGCGCGGCGCGGGGCTTTGTGCGCTCCGCAGTGTGCGCG
AGGGGAGCGCGGCCGGGGGCGGTGCCCCGCGGTGCGGGGGGGGCTGCGAG
GGGAACAAAGGCTGCGTGCGGGGTGTGTGCGTGGGGGGGTGAGCAGGGGG
TGTGGGCGCGTCGGTCGGGCTGCAACCCCCCCTGCACCCCCCTCCCCGAG
TTGCTGAGCACGGCCCGGCTTCGGGTGCGGGGCTCCGTACGGGGCGTGGC
GCGGGGCTCGCCGTGCCGGGCGGGGGGTGGCGGCAGGTGGGGGTGCCGGG
CGGGGCGGGGCCGCCTCGGGCCGGGGAGGGCTCGGGGGAGGGGCGCGGCG
GCCCCCGGAGCGCCGGCGGCTGTCGAGGCGCGGCGAGCCGCAGCCATTGC
CTTTTATGGTAATCGTGCGAGAGGGCGCAGGGACTTCCTTTGTCCCAAAT
CTGTGCGGAGCCGAAATCTGGGAGGCGCCGCCGCACCCCCTCTAGCGGGC
GCGGGGCGAAGCGGTGCGGCGCCGGCAGGAAGGAAATGGGCGGGGAGGGC
CTTCGTGCGTCGCCGCGCCGCCGTCCCCTTCTCCCTCTCCAGCCTCGGGG
CTGTCCGCGGGGGGACGGCTGCCTTCGGGGGGGACGGGGCAGGGCGGGGT
TCGGCTTCTGGCGTGTGACCGGCGGCTCTAGAGCCTCTGCTAACCATGTT
CATGCCTTCTTCTTTTTCCTACAGCTCCTGGGCAACGTGCTGGTTATTGT
GCTGTCTCATCATTTTGGCAAA

| Type | Start | End | Description |
|---|---|---|---|
| misc_feature | 1 | 1672 | CAG promoter |
| regulatory | 22 | 327 | CMV enhancer |
| promoter | 328 | 605 | chicken beta-actin promoter |
| intron | 607 | 1624 | chimeric intron/note = chimera |
| | | | between introns from chicken beta- |
| | | | actin and rabbit beta-globin |
| regulatory | 1528 | 1672 | predicted transcription factor sites |
| regulatory | 1575 | 1672 | predicted transcription factor binding site |

What is claimed:

1. A method for analyzing for or detecting the presence of enhancers of adeno-associated virus (AAV) vector cell transduction in a biological sample from a subject, comprising:
(a) providing infectious recombinant AAV particles comprising a recombinant AAV vector, wherein
(i) said vector comprises a reporter transgene,
(ii) said reporter transgene comprises a single-stranded or a self-complementary genome,
(iii) said reporter transgene is operably linked to one or more expression regulatory element; and
(iv) said reporter transgene is flanked by one or more flanking element;
(b) providing cells that can be infected with said infectious recombinant AAV particles;
(c) contacting said cells of (b) with said infectious recombinant AAV particles of (a) under conditions in which said cells of (b) are transduced by said infectious recombinant AAV particles of (a) and said reporter transgene is expressed by said cells of (b);
(d) measuring expression of said reporter transgene and assigning a value denoted MAX that reflects the amount of reporter transgene expression of (c);
(e) providing infectious recombinant AAV particles of (a);
(f) providing a biological sample from a subject;
(g) providing cells that can be infected with said infectious recombinant AAV particles;
(h) mixing said biological sample of (f) with empty capsid AAV particles to produce a mixture (M) and incubating said M under conditions allowing said empty capsid AAV particles to bind to any AAV binding antibodies present in said biological sample;
(i) contacting said cells of (g) with said M and said infectious recombinant AAV particles of (e) under conditions in which said infectious recombinant AAV particles of (e) can transduce said cells of (g) and express said reporter transgene in said cells of (g);
(j) measuring expression of said reporter transgene and assigning a value denoted S.EV that reflects the amount of reporter transgene expression of (i);
(k) comparing said S.EV to said MAX, wherein if said S.EV is greater than said MAX the biological sample from said subject contains enhancers of AAV vector cell transduction.

2. A method for analyzing for or detecting the presence of inhibitors of adeno-associated virus (AAV) vector cell transduction in a biological sample from a subject, comprising:
(a) providing infectious recombinant AAV particles comprising a recombinant AAV vector, wherein
(i) said vector comprises a reporter transgene,
(ii) said reporter transgene comprises a single-stranded or a self-complementary genome,
(iii) said reporter transgene is operably linked to one or more expression regulatory element; and
(iv) said reporter transgene is flanked by one or more flanking element;
(b) providing cells that can be infected with said infectious recombinant AAV particles;
(c) contacting said cells of (b) with said infectious recombinant AAV particles of (a) under conditions in which said cells of (b) are transduced by said infectious recombinant AAV particles of (a) and said reporter transgene is expressed by said cells of (b);
(d) measuring expression of said reporter transgene and assigning a value denoted MAX that reflects the amount of reporter transgene expression of (c);
(e) providing infectious recombinant AAV particles of (a);
(f) providing a biological sample from a subject;
(g) providing cells that can be infected with said infectious recombinant AAV particles;
(h) mixing said biological sample of (f) with empty capsid AAV particles to produce a mixture (M) and incubating said M under conditions allowing said empty capsid AAV particles to bind to any AAV binding antibodies present in said biological sample;
(i) contacting said cells of (g) with said M and said infectious recombinant AAV particles of (e) under conditions allowing said infectious recombinant AAV particles of (e) to transduce said cells of (g) and express said reporter transgene in said cells of (g);
(j) measuring expression of said reporter transgene and assigning a value denoted S.EV that reflects the amount of reporter transgene expression of (i);
(k) comparing said S.EV to said MAX, wherein if said S.EV is less than said MAX the biological sample from the subject contains inhibitors of AAV vector cell transduction, expression or secretion of a protein encoded by said vector.

3. A method for analyzing for or detecting the presence of enhancers of adeno-associated virus (AAV) vector cell transduction in a biological sample from a subject, comprising:
(a) providing infectious recombinant AAV particles comprising a recombinant AAV vector, wherein
(i) said vector comprises a reporter transgene,
(ii) said reporter transgene comprises a single-stranded or a self-complementary genome,
(iii) said reporter transgene is operably linked to one or more expression regulatory element; and
(iv) said reporter transgene is flanked by one or more flanking element;
(b) providing cells that can be infected with said infectious recombinant AAV particles;
(c) providing a biological sample from a subject;
(d) mixing said biological sample of (c) with empty capsid AAV particles to produce a mixture (M) and incubating said M under conditions allowing said empty capsid AAV particles to bind to any AAV binding antibodies present in said biological sample;
(e) contacting said cells of (b) with said M and said infectious recombinant AAV particles of (a) under conditions allowing said infectious recombinant AAV particles of (a) to transduce said cells of (b) and express said reporter transgene in said cells of (b);
(f) measuring expression of said reporter transgene;
(g) comparing said expression of (f) to a positive (+) control, wherein said + control is the expression of said reporter transgene without said sample from said subject and without addition of said empty capsid AAV particles, wherein if expression of (f) is greater than said + control, said biological sample from said subject contains enhancers of AAV vector cell transduction.

4. A method for analyzing for or detecting the presence of inhibitors of adeno-associated virus (AAV) vector cell transduction in a biological sample from a subject, comprising:
(a) providing infectious recombinant AAV particles comprising a recombinant AAV vector, wherein said vector comprises a reporter transgene,
(ii) said reporter transgene comprises a single-stranded or a self-complementary genome,
(iii) said reporter transgene is operably linked to one or more expression regulatory element, and
(iv) said reporter transgene is flanked by one or more flanking element;
(b) providing cells that can be infected with said infectious recombinant AAV particles;
(c) providing a biological sample from a subject;
(d) mixing said biological sample of (c) with empty capsid AAV particles to produce a mixture (M) and incubating said M under conditions allowing said empty capsid AAV particles to bind to any AAV binding antibodies present in said biological sample;
(e) contacting said cells of (b) with said M and said infectious recombinant AAV particles of (a) under conditions allowing said infectious recombinant AAV particles of (a) to transduce said cells of (b) and express said reporter transgene in said cells of (b);
(f) measuring expression of said reporter transgene;
(g) comparing said expression of (f) to a positive (+) control, wherein said + control is the expression of said reporter transgene without said sample from the subject and without addition of said empty capsid AAV particles, wherein if expression of (f) is less than said + control, said biological sample from said subject contains inhibitors of AAV vector cell transduction.

5. A method for analyzing for, detecting or quantifying AAV binding antibodies that inhibit AAV vector cell transduction in a biological sample from a subject, comprising:
(a) providing infectious recombinant AAV particles comprising a recombinant AAV vector, wherein
(i) said vector comprises a reporter transgene,
(ii) said reporter transgene comprises a single-stranded or a self-complementary genome,
(iii) said reporter transgene is operably linked to one or more expression regulatory element; and
(iv) said reporter transgene is flanked by one or more flanking element;
(b) providing cells that can be infected with said infectious recombinant AAV particles;
(c) contacting said cells of (b) with said infectious recombinant AAV particles of (a) under conditions in which said cells of (b) are transduced by said infectious recombinant AAV particles of (a) and said reporter transgene is expressed by said cells of (b);
(d) measuring expression of said reporter transgene and assigning a value denoted MAX that reflects the amount of reporter transgene expression of (c);
(e) providing infectious recombinant AAV particles of (a);
(f) providing a diluted biological sample from a subject;
(g) providing cells that can be infected with said infectious recombinant AAV particles;
(h) mixing said diluted biological sample of (f) with empty capsid AAV particles to produce a mixture (M) and incubating said M under conditions allowing said empty capsid AAV particles to bind to any AAV binding antibodies present in said diluted biological sample;
(i) contacting said cells of (g) with said M and said infectious recombinant AAV particles of (e) under conditions allowing said infectious recombinant AAV particles of (e) to transduce said cells of (g) and express said reporter transgene in said cells of (g);
(j) measuring expression of said reporter transgene and assigning a value denoted S.EV that reflects the amount of reporter transgene expression of (i);
(k) providing infectious recombinant AAV particles of (a);
(l) providing a diluted biological sample from the same subject that provided said sample of (f);
(m)providing cells that can be infected with said infectious recombinant AAV particles;
(n) mixing said diluted biological sample of (l) with said infectious recombinant AAV particles of (k) to produce a mixture (M);
(o) contacting said cells of (m) with said M under conditions allowing said infectious recombinant AAV particles of (k) to transduce said cells of (m) and express said reporter transgene in said cells of (m);
(p) measuring expression of said reporter transgene and assigning a value denoted S that reflects the amount of reporter transgene expression of (o);
(q) performing steps (h)-(j) and (n)-(p) at least twice at different sample dilutions;
(r) wherein if S is less than MAX and S.EV is equal to or greater than MAX, AAV binding antibodies that inhibit AAV vector cell transduction are present in said diluted biological sample; and, optionally
(s) measuring expression of a negative control of cells that can be infected with said infectious recombinant AAV particles of (a) but are not infected with infectious recombinant AAV particles of (a) to provide a background value denoted MIN, wherein MIN may be subtracted from any one of S, MAX and/or S.EV.

6. The method of claim 5, further comprising after step (r) or after step (s), calculating the titer of AAV binding antibodies in said biological sample, said titer corresponding to the lowest dilution of said biological sample that provides about 50% or more inhibition of reporter transgene expression, wherein if the lowest dilution that provides about 50% or more inhibition of reporter transgene expression is greater than or equal to about 1:5, said titer is the dilution that provides the about 50% or more inhibition determined by the formula S/MAX, or wherein if the lowest dilution that provides about 50% or more inhibition of reporter transgene expression is less than about 1:5, said titer is the dilution that provides the about 50% or more inhibition determined by the formula S/S.EV.

7. The method of claim 5, further comprising step (t), calculating the titer of AAV binding antibodies in said biological sample, said titer corresponding to the lowest dilution of said biological sample that provides about 50% or more inhibition of reporter transgene expression, wherein if the lowest dilution that provides about 50% or more inhibition of reporter transgene expression is greater than or equal to about 1:5, said titer is the dilution that provides the about 50% or more inhibition determined by the formula 100−[[(S−MIN)/(MAX−MIN)]×100], or wherein if the lowest dilution that provides about 50% or more inhibition of reporter transgene expression is less than about 1:5, said titer is the dilution that provides the about 50% or more inhibition determined by the formula 100−[[(S−MIN)/(S.EV−MIN)]×100].

8. The method of any one of claims 5-7, further comprising:
providing infectious recombinant AAV particles of (a);
providing cells that can be infected with said infectious recombinant AAV particles;
providing empty capsid AAV particles;
contacting said cells with said provided empty capsid AAV particles;
contacting said cells that have been contacted with said empty capsid AAV particles with said infectious recombinant AAV particles of (a) under conditions in which said cells of (b) are transduced by said infectious recombinant AAV particles of (a) and said reporter transgene is expressed by said cells;
measuring expression of said reporter transgene and assigning a value denoted MAX.EV that reflects the amount of reporter transgene expression.

9. The method of any one of claims 5-8, further comprising calculating a signal-to-noise ratio, denoted S/N, wherein S/N=MAX/MIN.

10. The method of any one of claims 5-9, further comprising calculating a percent coefficient of variation (% CV), wherein % CV=(standard deviation/mean)×100%.

11. The method of any one of claims 5-10, further comprising calculating EV interference, wherein EV interference=MAX/MAX.EV.

12. The method of any one of claims 5-12, further comprising:
(a) measuring expression of said reporter transgene under control conditions comprising one or more dilutions of AAV binding antibodies that bind to AAV vector, and assigning a value denoted HQC to said expression measurement of the dilution that provides a preselected amount of reporter transgene expression relative to MAX or MAX−MIN; and/or (b) measuring expression of said reporter transgene under control conditions comprising the dilution of step (a) that provides said preselected amount of reporter transgene expression relative to MAX or MAX−MIN in the presence of empty capsid AAV particles and assigning a value denoted HQC.EV.

13. The method of claim 12, wherein said preselected amount of reporter transgene expression of step (a) is equal to or less than about 30% of MAX or MAX−MIN.

14. The method of claim 12 or 13, further comprising calculating EV efficacy, wherein EV efficacy=HQC.EV/HQC.

15. The method of any one of claims 5-13, wherein steps (h)-(j) and (n)-(p) are performed at least 3, 4, 5 or 6 times at 3, 4, 5 or 6 different dilutions of said biological sample.

16. The method of any one of claims 5-14, wherein said biological sample is diluted between about 1:1 and about 1:1000 prior to contacting or incubating with said infectious recombinant AAV particles of (a), (e) or (k).

17. The method of any one of claims 5-16, wherein steps (h)-(j) and (n)-(p) are performed at about 1:1, about 1:2.5, about 1:5, about 1:10, about 1:100 and/or about 1:1000 dilutions of said biological sample.

18. The method of any one of claims 1-16, wherein said method is completed within about 48 hours of step (c) or (d).

19. The method of any one of claims 1-18, wherein said cells that can be infected with said infectious recombinant AAV particles are seeded from frozen cell aliquots.

20. The method of any one of claims 1-19, wherein said cells that can be infected with said infectious recombinant AAV particles of any of steps (c), (e), (i), or (o) are contacted within about 2 hours after said cells that can be infected with said infectious recombinant AAV particles are thawed from freezing.

21. The method of any one of claims 1-20, wherein said cells that can be infected with said infectious recombinant AAV particles of any of steps (c), (e), (i), or (o) are at least about 80% confluent.

22. A carrier or plate having individually disposed thereon:
(a) cells that can be infected with infectious recombinant AAV particles comprising a reporter transgene, and said infectious recombinant AAV particles comprising said reporter transgene;
(b) a diluted biological sample from a subject, cells that can be infected with infectious recombinant AAV particles, and empty capsid AAV particles; and
(c) a diluted biological sample from the same subject that provided the sample of (b), cells that can be infected with infectious recombinant AAV particles comprising a reporter transgene, and said infectious recombinant AAV particles comprising said reporter transgene.

23. The carrier or plate of claim 22, wherein said carrier or plate comprises plastic or glass.

24. The carrier or plate of claim 22 or 23, wherein said carrier or plate is a multiwell plate or dish.

25. The carrier or plate of any one of claims 22-24, wherein each of (a), (b) and (c) are disposed within a separate tube or a separate single well of a multiwell carrier or plate.

26. The method of any one of claims 1-21 or carrier or plate of any one of claims 22-25, wherein said cells that can be infected with said infectious recombinant AAV particles comprise mammalian cells.

27. The method of any one of claims 1-21 or carrier or plate of any one of claims 22-25, wherein said cells can be infected with AAV particles comprising a VP1, VP2 or VP3 sequence 90% or more identical to a VP1, VP2 or VP3 sequence of AAV serotype AAV1, AAV2, AAV3, AAV4, AAVS, AAV6, AAV7, AAV8, AAV9, AAV10, AAV11, AAV12, AAV3B, AAV-2i8, Rh74, Rh10, SPK1 (SEQ ID NO:1), SPK2 (SEQ ID NO:2) or a hybrid or chimera of any one of the foregoing AAV serotypes.

28. The method of any one of claims 1-21 or carrier or plate of any one of claims 22-25, wherein said cells can be infected with AAV serotype AAV1, AAV2, AAV3, AAV4, AAVS, AAV6, AAV7, AAV8, AAV9, AAV10, AAV11, AAV12, AAV3B, AAV-2i8, Rh74, Rh10, SPK1 (SEQ ID NO:1), SPK2 (SEQ ID NO:2), or a hybrid or chimera of any of the foregoing AAV serotypes.

29. The method of any one of claims 1-21 or carrier or plate of any one of claims 22-25, wherein said cells that can be infected with said infectious recombinant AAV particles comprise 2V6.11, HEK-293,CHO, BHK, MDCK, 10T1/2, WEHI cells, COS, BSC 1, BSC 40, BMT 10, VERO, WI38, MRCS, A549, HT1080, B-50, 3T3, NIH3T3, HepG2, Saos-2, Huh7, HER, HEK, HEL, or HeLa cells, optionally wherein any of said cells express the adenoviral E4 gene.

30. The method of any one of claims 1-21 or carrier or plate of any one of claims 22-25, wherein said reporter transgene encodes a secreted or secretable protein.

31. The method of any one of claims 1-21 or carrier or plate of any one of claims 22-25, wherein said reporter transgene encodes a protein that provides an enzymatic, colorimetric, fluorescent, luminescent, chemiluminescent, or electrochemical signal.

32. The method of any one of claims 1-21 or carrier or plate of any one of claims 22-25, wherein said reporter transgene comprises a luciferase gene.

33. The method of any one of claims 1-21 or carrier or plate of any one of claims 22-25, wherein said reporter trangene comprises a Renilla luciferase, a firefly luciferase, or a Gaussia luciferase gene.

34. The method of any one of claims 1-21 or carrier or plate of any one of claims 22-25, wherein said reporter transgene comprises a β-galactosidase gene, a β-glucoronidase gene, or a chloramphenicol transferase gene.

35. The method of any one of claims 1-21 or carrier or plate of any one of claims 22-25, wherein said reporter transgene encodes a green fluorescent protein (GFP), a red fluorescent protein (RFP) or an alkaline phosphatase.

36. The method of any one of claims 1-21 or carrier or plate of any one of claims 22-25, wherein said expression regulatory element comprises a promoter and/or enhancer nucleic acid sequence operable in mammalian cells.

37. The method of any one of claims 1-21 or carrier or plate of any one of claims 22-25 wherein said expression regulatory element comprises a CAG (SEQ ID NO:3), cytomegalovirus (CMV) immediate early promoter/enhancer, the Rous sarcoma virus (RSV) promoter/enhancer, SV40 promoter, dihydrofolate reductase (DHFR) promoter, chicken β-actin (CBA) promoter, phosphoglycerol kinase (PGK) promoter, or elongation factor-1 alpha (EF1-alpha) promoter.

38. The method of any one of claims 1-21 or carrier or plate of any one of claims 22-25, wherein said flanking element(s) comprises one or more AAV inverted terminal repeat sequences (ITRs).

39. The method of any one of claims 1-21 or carrier or plate of any one of claims 22-25, wherein said reporter transgene is positioned between one or more 5′ and/or 3′ AAV ITRs.

40. The method of any one of claims 1-21 or carrier or plate of any one of claims 22-25, wherein the flanking element(s) comprises a mutated, modified or variant AAV ITR that is not processed by AAV Rep protein.

41. The method of any one of claims 1-21 or carrier or plate of any one of claims 22-25, wherein said flanking element(s) comprises a mutated, modified or variant AAV ITR that allows or facilitates formation of the self-complementary reporter transgene genome into a double strand inverted repeat sequence structure in the infectious recombinant AAV particles.

42. The method of any one of claims 1-21 or carrier or plate of any one of claims 22-25, wherein said mutated, modified or variant AAV ITR has a deleted D sequence, and/or a mutated, modified or variant terminal resolution site (TRS) sequence.

43. The method of any one of claims 1-21 or carrier or plate of any one of claims 22-25, wherein said recombinant vector comprises a first inverted terminal repeat (ITR) of an AAV; a promoter operable in mammalian cells; said reporter transgene; a polyadenylation signal; and optionally a second ITR of an AAV.

44. The method of any one of claims 1-21 or carrier or plate of any one of claims 22-25, wherein said recombinant vector comprises a restriction site to allow insertion of said reporter transgene downstream of a promoter operable in mammalian cells, and a posttranscriptional regulatory element downstream of said restriction site, wherein said promoter, said restriction site and said posttranscription regulatory element are located downstream of a 5′ AAV ITR and upstream of an optional 3′ AAV ITR.

45. The method of any one of claims 1-21 or carrier or plate of any one of claims 22-25, wherein said infectious recombinant AAV particles comprise an AAV serotype that infects primates.

46. The method of any one of claims 1-21 or carrier of plate of any one of claims 22-25, wherein said infectious recombinant AAV particles comprise an AAV serotype that infects humans or rhesus macaques.

47. The method of any one of claims 1-21 or carrier or plate of any one of claims 22-25, wherein said infectious recombinant AAV particles comprise a VP1, VP2 or VP3 sequence 90% or more identical to a VP1, VP2 or VP3 sequence of AAV serotype AAV1, AAV2, AAV3, AAV4, AAVS, AAV6, AAV7, AAV8, AAV9, AAV10, AAV11, AAV12, AAV3B, AAV-2i8, Rh74, Rh10, SPK1 (SEQ ID NO:1), SPK2 (SEQ ID NO:2), or a hybrid or chimera of any of the foregoing AAV serotypes.

48. The method of any one of claims 1-21 or carrier or plate of any one of claims 22-25, wherein said infectious recombinant AAV particles comprise AAV serotype AAV1, AAV2, AAV3, AAV4, AAVS, AAV6, AAV7, AAV8, AAV9, AAV10, AAV11, AAV12, AAV3B, AAV-2i8, Rh74, Rh10, SPK1 (SEQ ID NO:1), SPK2 (SEQ ID NO:2), or a hybrid or chimera of any of the foregoing AAV serotypes.

49. The method of any one of claims 1-21 or carrier or plate of any one of claims 22-25, wherein said biological sample comprises a primate sample.

50. The method of any one of claims 1-21 or carrier or plate of any one of claims 22-25, wherein said biological sample comprises serum.

51. The method of any one of claims 1-21 or carrier or plate of any one of claims 22-25, wherein said biological sample comprises plasma.

52. The method of any one of claims 1-21 or carrier or plate of any one of claims 22-25, wherein said biological sample comprises human serum or human plasma.

53. The method of any one of claims 1-21 or carrier or plate of any one of claims 22-25, wherein said subject is a mammal.

54. The method of any one of claims 1-21 or carrier or plate of any one of claims 22-25, wherein said subject is a human, said human optionally afflicted with a heritable disease amenable to treatment by gene therapy.

55. The method of any one of claims 1-21 or carrier or plate of any one of claims 22-25, wherein said subject suffers from a disorder due to insufficient expression or activity of a protein.

56. The method of any one of claims 1-21 or carrier or plate of any one of claims 22-25, wherein said subject suffers from a disorder due to expression or activity of an abnormal, aberrant or undesirable protein.

57. The method of any one of claims 1-21 or carrier or plate of any one of claims 22-25, wherein said subject suffers from a genetic disorder.

58. The method of any one of claims 1-21 or carrier or plate of any one of claims 22-25, wherein said subject is a candidate for gene replacement or supplement therapy.

59. The method of any one of claims 1-21 or carrier or plate of any one of claims 22-25, wherein said subject is a candidate for gene knockdown or knockout therapy.

60. The method of any one of claims 1-21 or carrier of plate of any one of claims 22-25, wherein said subject suffers from a lung disease (e.g., cystic fibrosis), a bleeding disorder (e.g., hemophilia A or hemophilia B with or without inhibitors), thalassemia, a blood disorder (e.g., anemia), Alzheimer's disease, Parkinson's disease, Huntington's disease, amyotrophic lateral sclerosis (ALS), epilepsy, a lysosomal storage disease (e.g., aspartylglucosaminuria, Batten disease, cystinosis, late infantile neuronal ceroid lipofuscinosis type 2 (CLN2), Fabry disease, Gaucher disease types I, II, and III, glycogen storage disease II (Pompe disease), GM2-gangliosidosis type I (Tay Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A,B,C,D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorders (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), a retinal degenerative disease (e.g., RPE65 deficiency, choroideremia, Stargardt disease, and other diseases of the eye), a disease of solid organs (e.g., brain, liver, kidney, heart), or an infectious viral (e.g., hepatitis B and C, HIV, etc.), bacterial or fungal disease.

61. The method of any one of claims 1-21 or carrier or plate of any one of claims 22-25, further comprising entering information concerning the presence of or titer of AAV binding antibodies in said biological sample into a report associated with said subject.

62. The method of any one of claims 1-21 or carrier or plate of any one of claims 22-25, further comprising entering information concerning the presence of or titer of AAV binding antibodies in said biological sample into a database thereby producing a database entry, said database entry associated with said subject.

63. The method of any one of claims 1-21 or carrier or plate of any one of claims 22-25, wherein said AAV binding antibodies analyzed or detected bind to AAV serotype AAV1, AAV2, AAV3, AAV4, AAVS, AAV6, AAV7, AAV8, AAV9, AAV10, AAV11, AAV12, AAV3B, AAV-2i8, Rh74, or Rh10, or a hybrid or chimera of any of the foregoing AAV serotypes.

64. The method of any one of claims 1-21 or carrier or plate of any one of claims 22-25, wherein said biological sample is diluted prior to contacting with said infectious recombinant AAV particles of (a), (e) or (k).

65. The method of any one of claims 1-21 or carrier or plate of any one of claims 22-25, wherein a plurality of dilutions of said biological sample is analyzed.

66. The method of any one of claims 1-21 or carrier or plate of any one of claims 22-25, wherein a plurality of different dilution ratios of said biological sample is analyzed.

67. The method of any one of claims 1-21 or carrier or plate of any one of claims 22-25, wherein said cells that can be infected with said infectious recombinant AAV particles are not lysed prior to measuring expression of said reporter transgene.

68. The method of any one of claims 1-21 or carrier or plate of any one of claims 22-25, wherein said biological sample is heat inactivated.

69. The method of claim 1, wherein one or more of steps (c), (d), (h), (i), (j), or (k) is performed with an automated system.

70. The method of claim 69, wherein said automated system comprises contacting components, measuring components, mixing components, incubating components, a processor, and non-transitory electronic storage, said non-transitory electronic storage configured to cause said processor to control said contacting components, said measuring components, said mixing components, and said incubating components, said method further comprising:
(c) contacting, with said contacting components, the cells of (b) with said infectious recombinant AAV particles of (a) under the conditions in which said cells of (b) are transduced by said infectious recombinant AAV particles of (a) and said reporter transgene is expressed by said cells of (b);
(d) measuring, with said measuring components, the expression of said reporter transgene, assigning, with said processor, said value denoted MAX that reflects the amount of reporter transgene expression of (c), and storing, with said processor, said value denoted MAX in said non-transitory electronic storage;
(h) mixing, with said mixing components, said biological sample of (f) with said empty capsid AAV particles to produce said mixture M and incubating, with said incubating components, said M under the conditions allowing said empty capsid AAV particles to bind to any AAV binding antibodies present in said biological sample that inhibits, reduces or decreases AAV vector cell transduction;
(i) contacting, with said contacting components, said cells of (g) with said M and said infectious recombinant AAV particles of (e) under the conditions in which said infectious recombinant AAV particles of (e) can transduce said cells of (g) and express said reporter transgene in said cells of (g);
(j) measuring, with said measuring components, the expression of said reporter transgene, assigning, with said processor, said value denoted S.EV that reflects the amount of reporter transgene expression of (i), and storing, with said processor, said value denoted S.EV in said non-transitory electronic storage; and
(k) comparing, with said processor, said S.EV to said MAX, wherein if said S.EV is greater than said MAX, said processor determines said biological sample from said subject contains enhancers of AAV vector cell transduction, and optionally outputting, with said processor, an indication that said biological sample from said subject contains enhancers for display.

71. The method of claim 2, wherein one or more of steps (c), (d), (h), (i), (j), or (k) is performed with an automated system.

72. The method of claim 71, wherein said automated system comprises contacting components, measuring components, mixing components, incubating components, a processor, and non-transitory electronic storage, said non-transitory electronic storage configured to cause said processor to control said contacting components, said measuring components, said mixing components, and said incubating components, said method further comprising:
(c) contacting, with said contacting components, said cells of (b) with said infectious recombinant AAV particles of (a) under conditions in which said cells of (b) are transduced by said infectious recombinant AAV particles of (a) and said reporter transgene is expressed by said cells of (b);
(d) measuring, with said measuring components, expression of said reporter transgene, assigning, with said processor, said value denoted MAX that reflects the amount of reporter transgene expression of (c), and storing, with said processor, said value denoted MAX in the non-transitory electronic storage;
(h) mixing, with said mixing components, said biological sample of (f) with said empty capsid AAV particles to produce said mixture M and incubating, with said incubating components, said M under conditions allowing said empty capsid AAV particles to bind to any AAV binding antibodies present in said biological sample that inhibits, reduces or decreases AAV vector cell transduction;
(i) contacting, with said contacting components, said cells of (g) with said M and said infectious recombinant AAV particles of (e) under conditions allowing said infectious recombinant AAV particles of (e) to transduce said cells of (g) and express said reporter transgene in said cells of (g);
(j) measuring, with said measuring components, expression of said reporter transgene, assigning, with said processor, said value denoted S.EV that reflects the amount of reporter transgene expression of (i), and storing, with said processor, said value denoted S.EV in the non-transitory electronic storage; and
(k) comparing, with said processor, said S.EV to said MAX, wherein if said S.EV is less than said MAX said processor determines said biological sample from said subject contains inhibitors of AAV vector cell transduction, expression or secretion of a protein encoded by said vector, and optionally outputting, with said processor, an indication that said biological sample from said subject contains said inhibitors for display.

73. The method of claim 3, wherein one or more of steps (d), (e), (f), or (g) is performed with an automated system.

74. The method of claim 73, wherein said automated system comprises contacting components, measuring components, mixing components, incubating components, a processor, and non-transitory electronic storage, said non-transitory electronic storage configured to cause said processor to control said contacting components, said measuring components, said mixing components, and sad incubating components, said method further comprising:
(d) mixing, with said mixing components, said biological sample of (c) with said empty capsid AAV particles to produce said mixture M and incubating, with said incubating components, said M under conditions allowing said empty capsid AAV particles to bind to any AAV binding antibodies present in said biological sample that inhibits, reduces or decreases AAV vector cell transduction;
(e) contacting, with said contacting components, said cells of (b) with said M and said infectious recombinant AAV particles of (a) under conditions allowing said infectious recombinant AAV particles of (a) to transduce said cells of (b) and express said reporter transgene in said cells of (b);
(f) measuring, with said measuring components, expression of said reporter transgene; and
(g) comparing, with said processor, said expression of (f) to said positive (+) control, wherein if said expression of (f) is greater than said + control said processor determines said biological sample from said subject contains enhancers of AAV vector cell transduction, expression or secretion of a protein encoded by said vector, and optionally outputting, with said processor, an indication that said biological sample from said subject contains said enhancers for display.

75. The method of claim 4, wherein one or more of steps (d), (e), (f), or (g) is performed with an automated system.

76. The method of claim 75, wherein said automated system comprises contacting components, measuring components, mixing components, incubating components, a processor, and non-transitory electronic storage, said non-transitory electronic storage configured to cause said processor to control said contacting components, said measuring components, said mixing components, and said incubating components, said method further comprising:
(d) mixing, with said mixing components, said biological sample of (c) with said empty capsid AAV particles to produce said mixture M and incubating, with said incubating components, said M under conditions allowing said empty capsid AAV particles to bind to any AAV binding antibodies present in said biological sample that inhibits, reduces or decreases AAV vector cell transduction;
(e) contacting, with said contacting components, said cells of (b) with said M and said infectious recombinant AAV particles of (a) under conditions allowing said infectious recombinant AAV particles of (a) to transduce said cells of (b) and express said reporter transgene in said cells of (b);
(f) measuring, with said measuring components, expression of said reporter transgene; and
(g) comparing, with said processor, said expression of (f) to said positive (+) control, wherein if said expression of (f) is less than said + control said processor determines said biological sample from said subject contains inhibitors of AAV vector cell transduction, expression or secretion of a protein encoded by said vector, and optionally outputting, with said processor, an indication that said biological sample from said subject contains said inhibitors for display.

77. The method of claim 5, wherein one or more of steps (c), (d), (h), (i), (j), (n), (o), (p), or (s) is performed with an automated system.

78. The method of claim 77, wherein said automated system comprises contacting components, measuring components, mixing components, incubating components, a processor, and non-transitory electronic storage, said non-transitory electronic storage configured to cause said processor to control said contacting components, said measuring components, said mixing components, and said incubating components, said method further comprising:
(c) contacting, with said contacting components, said cells of (b) with said infectious recombinant AAV particles of (a) under conditions in which said cells of (b) are transduced by said infectious recombinant AAV particles of (a) and said reporter transgene is expressed by said cells of (b);
(d) measuring, with said measuring components, said expression of said reporter transgene, assigning, with said processor, said value denoted MAX that reflects the amount of reporter transgene expression of (c), and storing, with said processor, said value denoted MAX in said non-transitory electronic storage;
(h) mixing, with said mixing components, said diluted biological sample of (f) with said empty capsid AAV particles to produce said mixture M and incubating, with said incubating components, said M under conditions allowing said empty capsid AAV particles to bind to any AAV binding antibodies present in said biological sample that inhibits, reduces or decreases AAV vector cell transduction;
(i) contacting, with said contacting components, said cells of (g) with said M and said infectious recombinant AAV particles of (e) under conditions allowing said infectious recombinant AAV particles of (e) to transduce said cells of (g) and express said reporter transgene in said cells of (g);
(j) measuring, with said measuring components, said expression of said reporter transgene, assigning, with said processor, said value denoted S.EV that reflects the amount of reporter transgene expression of (i), and storing, with said processor, said value denoted S.EV in said non-transitory electronic storage;
(n) mixing, with said mixing components, said diluted biological sample of (l) with said infectious recombinant AAV particles of (e) to produce said mixture M;
(o) contacting, with said contacting components, said cells of (m) with said M under conditions allowing said infectious recombinant AAV particles of (k) to transduce said cells of (m) and express said reporter transgene in said cells of (m);
(p) measuring, with said measuring components, expression of said reporter transgene, assigning, with said processor, said value denoted S that reflects the amount of reporter transgene expression of (o), and storing, with said processor, said value denoted S in said non-transitory electronic storage; and
(s) optionally measuring, with said measuring components, expression of said negative control of cells that can be infected with said infectious recombinant AAV particles (a) but are not infected with infectious recombinant AAV particles (a) to provide a background value denoted MIN, wherein MIN may be subtracted, by said processor, from any one of S, MAX and/or S.EV.

79. The method of claim 78, further comprising step (s) or (t),
calculating, with said processor, a titer of said AAV binding antibodies, said titer corresponding to the lowest dilution of said biological sample that provides about 50% or more inhibition of reporter transgene expression, wherein said processor is configured such that if the lowest dilution that provides about 50% or more inhibition of reporter transgene expression is greater or equal to about 1:5, said titer is determined by the formula S/MAX, or if the lowest dilution that provides about 50% or more inhibition of reporter transgene expression titer is less than about 1:5, then said titer is determined by the formula S/S.EV; and
optionally outputting, with said processor, an indication of said titer for display.

80. The method of claim 78, further comprising step (t),
calculating, with said processor, a titer of said AAV binding antibodies, said titer corresponding to the lowest dilution of said biological sample that provides about 50% or more inhibition of reporter transgene expression, wherein said processor is configured such that if said lowest dilution that provides about 50% or more inhibition of reporter transgene expression is greater or equal to about 1:5, said titer is determined by the formula 100−[[(S−MIN)/(MAX−MIN)]×100], or if said lowest dilution that provides about 50% or more inhibition of reporter transgene expression titer is less than about 1:5, then said titer is determined by the formula 100−[[(S−MIN)/(S.EV−MIN)]×100]; and
optionally outputting, with the processor, an indication of said titer for display.

81. The method of any one of claims 78-80, further comprising:
providing infectious recombinant AAV particles (a);
providing cells that can be infected with said infectious recombinant AAV particles;
providing empty capsid AAV particles;
contacting, with said contacting components, said cells with said provided empty capsid AAV particles;
contacting, with said contacting components, said cells that have been contacted with said empty capsid AAV particles with said infectious recombinant AAV particles of (a) under conditions in which said cells of (b) are transduced by said infectious recombinant AAV particles of (a) and said reporter transgene is expressed by said cells;
measuring, with said measuring components, expression of said reporter transgene, assigning, with said processor, a value denoted MAX.EV that reflects the amount of reporter transgene expression, and storing, with said processor, said value denoted MAX.EV in said non-transitory electronic storage.

82. The method of any one of claims 78-81, further comprising calculating, with said processor, a signal-to-noise ratio, denoted S/N, wherein said S/N equals MAX/MIN, storing, with said processor, S/N in said non-transitory electronic storage, and optionally outputting, with said processor, an indication of S/N for display.

83. The method of any one of claims 78-82, further comprising calculating, with said processor, a percent coefficient of variation (% CV), wherein % CV=(standard deviation/mean)×100%, storing, with said processor, % CV in said non-transitory electronic storage, and optionally outputting, with said processor, an indication of % CV for display.

84. The method of any one of claims 78-83, further comprising calculating, with said processor, EV interference, wherein EV interference=MAX/MAX.EV, storing, with said processor, EV interference in said non-transitory electronic storage, and optionally outputting, with said processor, an indication of EV interference for display.

85. The method of any one of claims 78-84, further comprising calculating, with said processor, HQC based upon the expression measurement of the dilution that provides a preselected amount of reporter transgene expression relative to MAX or MAX−MIN and/or HQC EV based upon expression of said reporter transgene under control conditions comprising the dilution of step (a) that provides said preselected amount of reporter transgene expression relative to MAX or MAX−MIN in the presence of empty capsid AAV particles and/or HQC EV/HQC, storing, with said processor, HQC and/or HQC EV AND/OR HQC EV/HQC in said non-transitory electronic storage, and optionally outputting, with said processor, an indication of HQC and/or HQC EV and/or HQC EV/HQC for display.
